(19) United States
(12) Patent Application Publication
(10) Pub. No.: US 2020/0291079 A1
(43) Pub. Date: Sep. 17, 2020

(54) ENGINEERING STRUCTURALLY DEFINED NON-SACCHARIDE GLYCOSAMINOGLYCAN MIMETICS VIA A POLYPROLINE SCAFFOLD

(71) Applicant: AGENCY FOR SCIENCE, TECHNOLOGY AND RESEARCH, Singapore (SG)

(72) Inventors: Song-Gil LEE, Singapore (SG); Teck Chuan LIM, Singapore (SG); Shuting CAI, Singapore (SG); Su Seong LEE, Singapore (SG); Siv Ly KHOU, Singapore (SG); Kah Chin Jerry TOH, Singapore (SG); Joo-Eun JEE, Singapore (SG)

(21) Appl. No.: 15/775,354

(22) PCT Filed: Nov. 11, 2016
(86) PCT No.: PCT/SG2016/050559

§ 371 Date: May 10, 2018

(30) Foreign Application Priority Data
Nov. 12, 2015 (SG) 10201509331Y
Mar. 17, 2016 (SG) 10201602104X

(51) Int. Cl. C07K 14/47; A61P 25/00; A61P 35/00; C07K 14/00

(52) U.S. Cl. CPC C07K 14/47; A61P 25/00; A61K 38/00; C07K 14/001; A61P 35/00

(57) ABSTRACT

A non-saccharide glycosaminoglycan mimetic molecule comprising a polyproline backbone and one or more non-saccharide molecules, wherein the non-saccharide molecules are bound to one or more prolines and/or proline derivatives on the polyproline backbone, is disclosed herein. In a preferred embodiment the non-saccharide molecule is a negatively charged sulfate. The invention also relates to methods of synthesizing said non-saccharide glycosaminoglycan mimetic molecule and the use of the molecules thereof in targeting cell adhesion molecules such as selectin.

### TECHNICAL FIELD

The present invention generally relates to methods of generating a non-saccharide glycosaminoglycan (GAG) mimetic using a polyproline scaffold, and the uses thereof to develop target-specific therapeutic agents and biomarkers. The present invention also relates to a method of generating non-saccharide glycosaminoglycan mimetics by controlling the display of pendant groups on a polyproline scaffold.

### BACKGROUND

Glycosaminoglycan (GAG) mimetics are compounds designed to recapitulate the structural and functional characteristics of glycosaminoglycan^{[1]}. With the exception of chemoenzymatic methods, a large proportion of glycosaminoglycan mimetic strategies either synthetically build oligosaccharides consisting of several glycosaminoglycan sugar units^{[2]} or assemble multivalent architectures by conjugating bioactive glycosaminoglycan carbohydrate moieties to synthetic scaffolds^{[3]}. These methods are time consuming, atom inefficient, costly and involve laborious carbohydrate synthesis, thus making glycosaminoglycan mimetics synthetically inaccessible and not economical. The complicated multi-step carbohydrate synthesis required for their generation has remained a critical problem that has hampered their widespread adoption^{[4]}. While sulfated, polyphenol-based non-saccharide glycosaminoglycan mimetics have been reported^{[4-5],} these polysulfated molecules are small molecules that are limited in their ability to mimic multivalent interactions of natural long glycosaminoglycan chains, which in turn limits their applicability.

One important area in which such glycosaminoglycan mimetics may be used is in drug design for treatment of diseases, such as cancer. Such application would require control and optimization of the efficiency and specificity of glycosaminoglycan mimetics towards binding certain molecular targets. Such molecular targets include cell adhesion molecules, such as selectins, integrins and receptor for advanced glycation end products (RAGE). Previously, the lack of means to systematically and precisely control the molecular structure and spatial display of epitopes on glycosaminoglycan mimetics have been a stumbling block in creating mimetics that follow the diverse, yet context-specific functions of natural glycosaminoglycans. It has also hindered the adoption of molecular structure and epitope display as a prominent design parameter for glycosaminoglycan mimetic-based drugs despite the common understanding that these attributes are instrumental in driving glycosaminoglycan-mediated functions.

Selectins (P-, L- and E-selectins) make up a family of glycoproteins that are commonly expressed on platelets, leukocytes and endothelial cells. Armed with a lectin domain that recognizes specific polysaccharide structures, selectins mediate cell adhesion events and play key roles in physiological processes such as constitutive leukocyte trafficking. Selectins are also implicated in several pathophysiological contexts such as cancer metastasis, inflammation and vaso-occlusion crisis in sickle cell disease. In particular for cancer metastasis (which has remained to be the main cause for morbidity and mortality among cancer patients), circulating tumor cells hijack the leukocyte trafficking mechanism by binding to selectins, extravasate and invade other tissues. For these reasons, there has been enormous interest in devising ways to inhibit selectin-based interactions.

Heparin, a natural glycosaminoglycan, has been a widely explored as an inhibitor of selectin-based interactions. Being the most highly sulfated glycosaminoglycan, heparin has been found to bind strongly with P- and L-selectin and to effectively disrupt interactions with their native ligands. Given that heparin is already a FDA-approved drug, inhibition of selectins by heparin can furthermore be readily translated into clinical applications if heparin can be verified as a successful candidate. Unfortunately, the intended use of heparin, which is to prevent blood coagulation, becomes its major drawback if heparin is to be used as a selectin inhibitor. Numerous attempts have been made to produce chemically modified, non-anti-coagulant heparin derivatives.^{[6-8]} However, such derivatives often inherit the chemical heterogeneity of naturally-derived heparin as well as the risk of contamination, which can induce severe anaphylactoid reactions similar to those reported in the previous contaminated-heparin scare.^{[9]}

There is a need to provide a method of synthesizing non-carbohydrate alternatives that can mimic glycosaminoglycan functions that overcomes, or at least ameliorates, one or more of the disadvantages described above.

There is a need to provide structurally well-defined, non-saccharide and multivalent glycosaminoglycan mimetics in which the spatial arrangement of pendant groups can be precisely controlled using a polyproline scaffold, for controlling and enhancing binding specificity to a target molecule. Each target molecule has its own unique structural confirmation, which in turn dictates the requirements of the spatial arrangement of the pendant groups of the glycosaminoglycan mimetic for achieving the optimal binding fit. Thus, there is a need to provide a method to tailor non-saccharide glycosaminoglycan mimetic molecules for binding to specific target molecules. There is also a need to provide a closer mimic to the longer natural glycosaminoglycan that also allows for a greater degree of control.

### SUMMARY

According to a first aspect, there is provided a non-saccharide glycosaminoglycan mimetic molecule comprising a polyproline backbone and one or more non-saccharide molecules. Advantageously, the non-saccharide glycosaminoglycan mimetic molecule is a well-defined, non-saccharide, multivalent glycosaminoglycan mimetic in which the spatial arrangement of the pendant groups has been precisely controlled using a polyproline scaffold, which allows for the use of this mimetic in controlling protein binding specificity and efficacy.

Non-saccharide molecules may be attached to one or more prolines and/or proline derivatives that make up the polyproline backbone. Advantageously, the non-saccharide pendant groups allow for controlling the specificity of these mimetics by precise positioning of non-saccharide bioactive moieties to direct multivalent interactions, allowing for a wide range of potential applications.

According to a second aspect, there is provided a method of synthesizing a non-saccharide glycosaminoglycan mimetic molecule as defined above, comprising attaching one or more non-saccharide molecules to a polyproline backbone. Advantageously, the non-carbohydrate alternative avoids the need for time consuming, atom inefficient, costly and laborious carbohydrate synthesis.

According to a third aspect, there is provided a non-saccharide glycosaminoglycan mimetic molecule as defined above for use in therapy. Advantageously, the use of the non-saccharide glycosaminoglycan mimetic molecule enables specific binding to a target molecule (for example a protein such as selectin) that is implicated in disease conditions, thereby avoiding undesirable consequences (such as side effects that may arise from unspecific cellular binding).

According to a fourth aspect, there is provided a method for inhibiting cell adhesion molecules comprising administering a non-saccharide glycosaminoglycan mimetic molecule as defined above. Advantageously, the non-saccharide glycosaminoglycan mimetic molecule has non-anti-coagulant property compared to heparin (which is used as an anti-coagulant itself) when used as a cell adhesion molecule inhibitor.

According to a fifth aspect, there is provided a method of treating a patient in need of a target-specific therapy, comprising administering a non-saccharide glycosaminoglycan mimetic molecule as defined above.

According to a sixth aspect, there is provided a target-specific therapeutic agent comprising a non-saccharide glycosaminoglycan mimetic molecule as defined above.

According to a seventh aspect, there is provided a non-saccharide glycosaminoglycan mimetic molecule as defined above for use as target-specific biopolymers.

According to an eighth aspect, there is provided a non-saccharide glycosaminoglycan mimetic molecule as defined above for use in glycosaminoglycans (GAG)-based pharmaceutics. According to a ninth aspect, there is provided a non-saccharide glycosaminoglycan mimetic molecule as defined above for use as diagnostic tools.

According to a tenth aspect, there is provided a method of controlling the binding affinity of a non-saccharide glycosaminoglycan mimetic molecule to one or more binding molecules, comprising attaching one or more non-saccharide molecules at pre-determined positions along a polyproline backbone. Advantageously, the precise positioning of non-saccharide molecules on the polypeptide backbone of the non-saccharide glycosaminoglycan mimetic molecule may be used to direct multivalent interactions in a more controlled and specific manner, allowing for a wide range of potential applications.

According to an eleventh aspect, there is provided a method of promoting neuritogenesis in a patient, comprising administering a non-saccharide glycosaminoglycan mimetic molecule as defined above.

Accordingly to a twelfth aspect there is provided a method of inhibiting extravasation of circulating tumor cell into potential metastatic sites in a patient, comprising administering a non-saccharide glycosaminoglycan mimetic molecule as defined above.

### Definition of Terms

The following words and terms used herein shall have the meaning indicated:

The term “glycosaminoglycan” refers to any complex polysaccharides having repeating units of either the same saccharide subunit or two different saccharide subunits. Some examples of natural glycosaminoglycans include dermatan sulfates, hyaluronic acid, the chondroitin sulfates, chitin, heparin, keratan sulfates, keratosulfates, heparan sulfates, and derivatives thereof.

The term “protein”, “polypeptide,” and “peptide” used interchangeably herein refers to a polymer of at least two amino acids that are covalently linked. The amino acids may be D- or L-amino acids, or mixtures of D- and L-amino acids, as well as naturally occurring or synthetically produced amino acids.

The term “mimetic” refers to a molecule that has a structure, and typically biological properties, that are similar to the molecule it is imitating. For example, when used with reference to a non-saccharide glycosaminoglycan molecule as defined herein, the term refers to a molecule which because of its structural properties, is capable of mimicking the biological function of a glycosaminoglycan.

The term “derivative” refers to a chemically or biologically modified version of a compound or molecule that is structurally similar to a parent compound or molecule and is derived from that parent compound or molecule.

The term “pendant group” refers to any functional group that may be attached to, and forms a side-chain of a macromolecule. Typically, the pendant group is attached to the backbone of the macromolecule. For example, in the case of a non-saccharide glycosaminoglycan mimetic, the non-saccharide molecule may form the pendant group that is attached to the polyproline backbone of the glycosaminoglycan mimetic, via linkages such as a covalent bond. Exemplary pendant groups on the polyproline backbone of a non-saccharide glycosaminoglycan mimetic include, but are not limited to, hydroxyl, sulfate, phosphate or carboxylate group-containing non-saccharides. The non-saccharide glycosaminoglycan mimetic may contain one of the hydroxyl, sulfate, phosphate or carboxylate group-containing non-saccharides, or a combination thereof.

The term “pre-determined,” for example, when used with reference to positions along the polyproline backbone of the glycosaminoglycan mimetic of the present disclosure, refers to any position along the polyproline backbone that has been selected for attachment of one or more non-saccharide molecules. The position(s) may, for example, have been selected for attachment of one or more non-saccharide molecules to modulate one or more biological functions of the glycosaminoglycan mimetic, for example improved molecular stability, improved selectivity or specificity, improved binding affinity, or the like.

The term “attach,” and variations of that term including “attaching” and “attachment,” refers to any form of association of one molecule to another, either directly or indirectly (such as via a linker), via any means including but not limited to a covalent bond, via hybridization, via non-covalent interactions, such as receptor-ligand interactions.

The term “treatment” includes any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment.

The term “alkyne-functionalized” refers to the incorporation of an alkyne functional group into a molecule, typically to facilitate subsequent chemical reaction to take place with or via the alkyne functional group.

The term “patient” refers to patients of human or other mammal and includes any individual it is desired to examine or treat using the methods of the disclosure. However, it will be understood that “patient” does not imply that symptoms are present. Suitable mammals that fall within the scope of the disclosure include, but are not restricted to, primates, livestock animals (eg. sheep, cows, horses, donkeys, pigs), laboratory test animals (eg. rabbits, mice, rats, guinea pigs, hamsters), companion animals (eg. cats, dogs) and captive wild animals (eg. foxes, deer, dingoes).

The term “administering” and variations of that term including “administer” and “administration”, includes contacting, applying, delivering or providing a compound or composition of the disclosure to an organism, or a surface by any appropriate means.

The term “target-specific” when used in relation to therapy such as in “target-specific therapy,” it is meant the administration of a compound, for example a drug (such as a glycosaminoglycan mimetic of the present disclosure), to a patient in need of therapy, that is capable of binding to a particular biological target to cause a desired biological or therapeutic effect on the patient in order to treat the patient. Similarly, “target-specific therapeutic agent” refers to a therapeutic agent that is specific to a particular target molecule or disease (for example a target-specific drug), while “target-specific biopolymer” refers to a biopolymer that binds to a particular biological target.

The term “face” as used herein with reference to the polyproline backbone, refers to a distinct surface on the polyproline backbone to which a pendant group may be attached. Several faces may run roughly parallel along the same polyproline backbone.

The term “inhibit” or “inhibitor” as used herein refers to a molecule that interferes (e.g. prevents) with the interaction (e.g. binding) between two or more other molecules. The inhibition can take place in vitro or in vivo, and can be a direct or indirect inhibition.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### Detailed Disclosure of the Embodiment

Exemplary, non-limiting embodiments of a non-saccharide glycosaminoglycan mimetic molecule, a method for synthesizing the non-saccharide glycosaminoglycan mimetic molecule, and uses of the non-saccharide glycosaminoglycan mimetic molecule, as well as a method of controlling the binding affinity of a non-saccharide glycosaminoglycan mimetic molecule to its binding molecules will now be disclosed.

In a first aspect, there is provided a non-saccharide glycosaminoglycan mimetic molecule comprising a polyproline backbone and one or more non-saccharide molecules. Each proline in the polyproline backbone may independently be a proline or a proline derivative. The proline derivative may comprise a functional group for conjugation to a non-saccharide molecule, and may be selected from the group consisting of azidoproline, aminoproline, mercaptoproline, prolinecarboxylic acid, hydroxyproline and enantiomers thereof. Exemplary proline derivatives include, but are not limited to, azidoproline, aminoproline, mercaptoproline, prolinecarboxylic acid, hydroxyproline, or enantiomers thereof, wherein the enantiomers include (4R)-azidoproline, (4R)-aminoproline, (4R)-mercaptoproline, (4R)-prolinecarboxylic acid, (4R)-hydroxyproline, (4S)-azidoproline, (4S)-aminoproline, (4S)-mercaptoproline, (4S)-prolinecarboxylic acid, and (4S)-hydroxyproline.

In one embodiment, the non-saccharide molecules comprise primary, secondary or tertiary negatively charged groups, or a combination thereof. The negatively charged groups may be selected from the group consisting of hydroxyl, sulfates, carboxylates and phosphates.

In one embodiment, the non-saccharide molecules have a structure selected from the group consisting of:

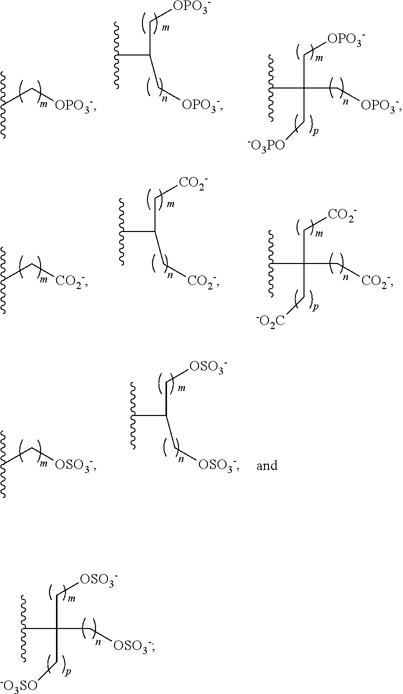

wherein m, n, and p are 0 or a positive integer greater than 1. In one embodiment, the non-saccharide molecule has the following structure:

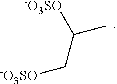

The non-saccharide molecules may be bound to one or more prolines and/or proline derivatives on the polyproline backbone.

In one embodiment, the polyproline backbone has the following general formula (I):

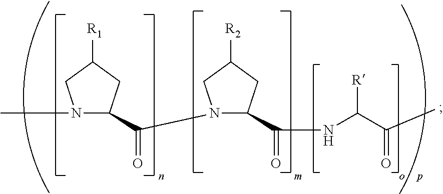

wherein R₁ and R₂ is H or a functional group for conjugation to a non-saccharide molecule; R′ is any amino acid side chain, n is a positive integer greater than 1; m is 0 or a positive integer, O is 0 or a positive integer, wherein at least R₁ or R₂ is a functional group for conjugation to a non-saccharide molecule, and p is a positive integer greater than 1. The n may be 1, 2, 3, 4, 5, 6, 7, 8, 9, 10, 11 or 12. The m may be 0, 1, 2, 3, 4, 5, 6, 7, 8, 9, 10, 11 or 12. In one embodiment, n is 1, m is 0, O is 0, R₁ is an azido group, and p is 12. In another embodiment, n is 2, m is 1, O is 0, R₁ is H, R₂ is azido, and p is 12. In yet another embodiment, n is 4, m is 0, and O is 1, R₁ is azido, R′ is H, and p is 3.

In one embodiment, R′ is any amino acid side chain other than proline.

In one embodiment, the polyproline backbone has the following formula (II):

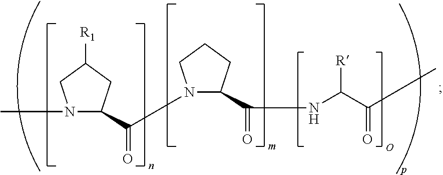

wherein R₁ is a functional group for conjugation to a non-saccharide molecule; R′ is any amino acid side chain; n is a positive integer greater than 1; m is 0 or a positive integer; O is 0 or a positive integer, and p is a positive integer greater than 1. In one embodiment, the functional group for conjugation to a non-saccharide molecule is N₃.

The polyproline backbone may be rigid or semi-flexible. The polyproline backbone may also comprise one or more glycine to allow the backbone to be semi-flexible.

The type of non-saccharide may be selected based on the biological target (for example, a protein) to be bound. For example, where the biological target is GDNF, GFRα1, or a selectin, a suitable non-saccharide for forming the non-saccharide glycosaminoglycan mimetic may be a non-saccharide sulfated mimetic (NS) that is designed to contain both a primary and secondary sulfation group that is incorporated into the PPII helix by click reaction. Other suitable non-saccharides may be used in place of NS. The person skilled in the art would be able to determine the type of non-saccharide that is suitable for forming the non-saccharide glycosaminoglycan mimetic in order to bind a desired biological target. The positioning of the non-saccharide(s) on the polyproline backbone may be determined based on factors such as the type and/or structure of the non-saccharide molecules used, and also the type and/or structure (e.g. the crystal structure) of the desired biological target.

In one embodiment, the non-saccharide molecules are attached at pre-determined positions along the polyproline backbone.

In one embodiment, the non-saccharide molecules are attached at equal distances along the polyproline backbone. For example, the pendant groups may be spaced at about 1, 2, 3, 4, 5, 6, 7, 8, 9, 10, or more A apart along the polyproline backbone. In another example, the pendant groups may be spaced at about 10, 20, 30, 40, 50, 60, 70, 80, 90, 100 or more A apart along the polyproline backbone. In another example, the pendant groups may be spaced at about between 2 to 100 Å, or 2 to 15 Å, or 8 to 30 Å, or 25 to 50 Δ, 45 to 70 Δ, 65 to 90 Å, or 85 to 110 Å apart along the polyproline backbone. In one embodiment, the pendant groups are spaced at 10 Å apart along the polyproline backbone.

In another embodiment, the non-saccharide molecules are attached along more than one different faces of the polyproline backbone. The non-saccharide molecules may be attached along three faces and project from the polyproline backbone. In another embodiment, the non-saccharide molecules are attached along the same face of the polyproline backbone. The inventors have advantageously found that the first distributed design demonstrated higher binding affinities to GDNF, GFRα1, and P-selectin when compared to the single-facial design containing the same number of NS moieties. The inventors have also advantageously found that the second single-facial design demonstrated higher binding affinity to L-selectin, when compared to the distributed design containing the same number of NS moieties. Therefore, it is possible to tailor and design the distribution of the non-saccharide molecules on the faces of the proline backbone depending on the target binding molecule.

In one embodiment, the non-saccharide glycosaminoglycan mimetic molecule further comprises polyethylene glycol (PEG) at one end of the polyproline backbone. The PEG may be biotin conjugated to facilitate surface attachment. The PEG may also be 1,2-dipalmitoyl-sn-glycero-3-phosphoethanolamine-N-(succinyl) conjugated to facilitate cell membrane insertion.

In one embodiment, the non-saccharide glycosaminoglycan mimetic molecule has the following general formula (III):

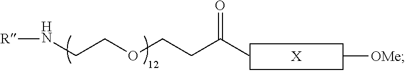

wherein R″ is any functional moiety, and wherein X is a non-saccharide glycosaminoglycan mimetic molecule as defined above. The R″ may be selected from the group consisting of H, 1,2-dipalmitoyl-sn-glycero-3-phosphoethanolamine-N-(succinyl)] and biotin. In one embodiment, X is (P_{E})₁₂, (PPP_{E})₁₂, or (P_{E})₄G(P_{E})₄G(P_{E})₄G wherein P is proline and P_{E} is

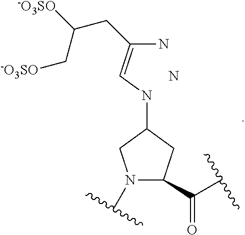

In one embodiment, the non-saccharide glycosaminoglycan mimetic molecule as defined above further comprises a lipid. Advantageously, the lipid allows the insertion of the non-saccharide glycosaminoglycan mimetic molecule into the lipid bilayer membrane surrounding a cell.

The non-saccharide glycosaminoglycan mimetic molecule as defined above may also comprise more than one non-saccharide molecules. The non-saccharide molecules may be hydroxyl, sulfate, phosphate or carboxylate group-containing non-saccharides, or a combination thereof. For example, all of the non-saccharide molecules may be hydroxyl group-containing non-saccharides, all of the non-saccharide molecules may be sulfate group-containing non-saccharides, or all of the non-saccharide molecules may be phosphate group-containing non-saccharides, or all of the non-saccharide molecules may be carboxylate group-containing non-saccharides. Alternatively, a non-saccharide glycosaminoglycan mimetic molecule may have a combination of a hydroxyl group-, sulfate group- and phosphate group-containing non-saccharides, or hydroxyl group-, sulfate group- and carboxylate group-containing non-saccharides, or hydroxyl group-, phosphate group- and carboxylate group-containing non-saccharides, or carboxylate group-, sulfate group- and phosphate group-containing non-saccharides. Alternatively, a non-saccharide glycosaminoglycan mimetic molecule may have a combination of hydroxyl group- and phosphate group-containing non-saccharides, hydroxyl group- and sulfate group-containing non-saccharides, hydroxyl group- and carboxylate group-containing non-saccharides, sulfate group- and phosphate group-containing non-saccharides, or sulfate group- and carboxylate group-containing non-saccharides, or phosphate group- and carboxylate group-containing non-saccharides, or hydroxyl group-, sulfate group-, phosphate group- and carboxylate group-containing non-saccharides.

In yet another embodiment, the non-saccharide glycosaminoglycan mimetic molecule as defined above is capable of binding cell adhesion molecules. Exemplary cell adhesion molecules include, but are not limited to, selectins, integrins, cadherins, addressins and Receptor for Advanced Glycation End Products (RAGE).

In a second aspect, there is provided a method of synthesizing a non-saccharide glycosaminoglycan mimetic molecule as defined above, comprising attaching one or more non-saccharide molecules to a polyproline backbone. Advantageously, the non-carbohydrate alternative overcomes the need for time consuming, atom inefficient, costly and laborious carbohydrate synthesis. The non-saccharide molecules may comprise sulfated groups. In one embodiment, the non-saccharide molecules have the structure of

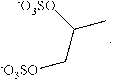

The non-saccharide molecule may also be alkyne-functionalized, while the polyproline backbone may be azido-functionalized.

In one embodiment of the method, the one or more non-saccharide molecules are attached to the polyproline backbone via click reaction. The click reaction may be conducted in dimethyl sulfoxide (DMSO) at ambient temperature for about 14 days in the presence of copper(I) idode, N,N-diisopropylethylamine (DIPEA) and tris[(1-benzyl-1H-1,2,3-triazol-4-yl)methyl]amine (TBTA) under argon atmosphere.

The method may comprise the steps of: (i) precipitating the reaction mixture resulting from the click reaction from a THF/methanol mixture and removing the solvent of the reaction mixture by vacuum and decanting the solid; (ii) converting the reaction mixture into their sodium salt form; and (iii) purifying the salt by size-exclusion chromatography.

The method may further comprise, prior to the click reaction: (a) conjugating PEG₁₂ to the polyproline backbone in the presence of N,N-diisopropylethylamine (DIPEA) base and Dimethylformamide (DMF) at room temperature, and optionally (b) coupling 1,2-dipalmitoyl-sn-glycero-3-phosphoethanolamine-N-(succinyl) sodium salt or biotin to the PEG₁₂ via amide coupling in the presence of benzotriazol-1-yl-oxytripyrrolidinophosphonium hexafluorophosphate(PyBOB), hydroxybenzotriazole (HOBt), N,N-diisopropylethylamine (DIPEA) base and Dimethylformamide (DMF) at room temperature.

In one embodiment, the non-saccharide molecule are alkyne-functionalized having the following structure:

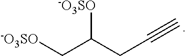

The alkyne-functionalized non-saccharide molecule may be synthesized by:
(i) reacting ethyl glyoxylate,

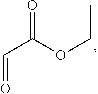

and propargyl bromide,

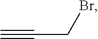

to form Ethyl 2-hydroxypent-4-ynoate,

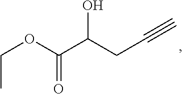

via a zinc Barbier reaction;
(ii) reducing the (Ethyl 2-hydroxypent-4-ynoate),

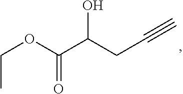

by lithium aluminum hydride to form Pent-4-yne-1,2-diol),

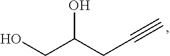

and
(iii) sulfating (Pent-4-yne-1,2-diol),

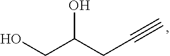

with an SO₃.trimethyllamine complex (Sulfur trioxide trimethylamine complex) to form (Pent-4-yne-1,2-diyl bis(sulfate),

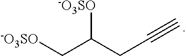

In one embodiment, the Barbier reaction in step (i) above is conducted in the presence of zinc (Zn), calcium chloride (CaCl₂), ammonium chloride (NH₄Cl) and tetrahydrofuran-water (THF-H₂O) at room temperature. In another embodiment, step (ii) above is conducted in the presence of tetrahydrofuran (THF) at room temperature. In yet another embodiment, the above step (iii) is conducted in the presence of dimethylformamide (DMF) at room temperature.

In a third aspect, there is provided a non-saccharide glycosaminoglycan mimetic molecule as defined above for use in therapy. The non-saccharide glycosaminoglycan mimetic molecule as defined above may be formulated into a suitable pharmaceutical composition for administration to a patient in need thereof. There is also provided a use of the non-saccharide glycosaminoglycan mimetic molecule as defined above in the manufacture of a medicament for therapy.

In a fourth aspect, there is provided a method of inhibiting cell adhesion molecules comprising administering a non-saccharide glycosaminoglycan mimetic molecule as defined above. In one embodiment, the method comprises inhibiting the cell adhesion molecule from binding one or more of its targets (e.g. tumor cells). There is also provided a non-saccharide glycosaminoglycan mimetic molecule as defined above for use in inhibiting cell adhesion molecules, as well as a use of the non-saccharide glycosaminoglycan mimetic molecule as defined above in the manufacture of a medicament for inhibiting cell adhesion molecules. The cell adhesion molecule may be selectin, integrins or receptor for advanced glycation end products (RAGE). In one embodiment, the cell adhesion molecule is selectin. In one embodiment, the selectin is inhibited (prevented) from binding to tumor cells; to thereby inhibit tumor metastasis.

In a fifth aspect, there is provided a method of treating a patient in need of a target-specific therapy, comprising administering a non-saccharide glycosaminoglycan mimetic molecule as defined above. There is also provided a use of the non-saccharide glycosaminoglycan mimetic molecule as defined above in the manufacture of a medicament for treating a patient in need of a target-specific therapy. Further provided are the non-saccharide glycosaminoglycan mimetic molecules as defined above for use in target-specific therapy. Exemplary target-specific therapy in which a non-saccharide glycosaminoglycan mimetic molecule as defined above may be useful includes, but is not limited to, cancer therapy, HIV-therapy, and therapy against diseases such as neurodegenerative diseases (e.g. Parkinson's disease, Alzheimer's disease, etc.), bone diseases, cartilage diseases, immunological diseases (e.g. rheumatoid arthritis, osteoporosis), inflammatory diseases, and infections such as bacterial infections, viral infections, and fungal infections. Other exemplary viral infections include, but are not limited to, dengue virus, herpes simplex virus, yellow fever virus, West Nile virus, hepatitis C virus, Chikungunya virus, respiratory syncytial virus, measles virus, and foot and mouth disease virus.

In a sixth aspect, there is provided a target-specific therapeutic agent comprising a non-saccharide glycosaminoglycan mimetic molecule as defined above. Exemplary target-specific therapeutic agents in which a non-saccharide glycosaminoglycan mimetic molecule as defined above may be useful include, but are not limited to, anti-cancer agents, anti-HIV agents, anti-inflammatory agents, anti-bacterial agents, anti-viral agents, anti-fungal agents, antibiotics, neuronal promoters, or the like.

In a seventh aspect, there is provided a non-saccharide glycosaminoglycan mimetic molecule as defined above for use as target-specific biopolymers. Exemplary target-specific biopolymers in which a non-saccharide glycosaminoglycan mimetic molecule as defined above may be useful include, but are not limited to, anticoagulants (such as anticoagulant heparin mimetics), modulators of physiological activity (such as modulators of chemokine activity with clinical relevance to diseases such as atherosclerosis, cancer, and autoimmune disorders), anti-dengue agents, anti-malarial agents, or the like. In one embodiment, the non-saccharide glycosaminoglycan mimetic molecule specifically targets cell adhesion molecules. In one embodiment, the cell adhesion molecule is a P-selectin or L-selectin. Exemplary cell adhesion molecules include, but are not limited to, selectins, integrins, cadherins, addressins and Receptor for Advanced Glycation End Products (RAGE).

In an eighth aspect, there is provided a non-saccharide glycosaminoglycan mimetic molecule as defined above for use in glycosaminoglycans (GAG)-based pharmaceutics. Exemplary GAG-based pharmaceutics in which a non-saccharide glycosaminoglycan mimetic as defined above may be useful include, but are not limited to, anticoagulants (such as anticoagulant heparin mimetics), modulators of physiological activity (such as modulators of chemokine activity with clinical relevance to diseases such as atherosclerosis, cancer, and autoimmune disorders), anti-dengue agents, anti-malarial agents, or the like.

In a ninth aspect, there is provided a non-saccharide glycosaminoglycan mimetic molecule as defined above for use as diagnostic tools. Exemplary diagnostic tools in which a non-saccharide glycosaminoglycan mimetic molecule as defined above may be useful include, but are not limited to, a diagnostic tool for cancer, infection (e.g. bacterial, viral or fungal infection), substance abuse, or the like.

In a tenth aspect, there is provided a method of controlling the binding affinity of a non-saccharide glycosaminoglycan mimetic molecule to one or more binding molecules, comprising attaching one or more non-saccharide molecules at pre-determined positions along a polyproline backbone. Each proline in the polyproline backbone may independently be a proline or a proline-derivative. In one embodiment, the proline derivative comprises a functional group for conjugation to a non-saccharide molecule. The proline derivative may be selected from the group consisting of azidoproline, aminoproline, mercaptoproline, prolinecarboxylic acid, hydroxyproline and enantiomers thereof. Exemplary proline derivatives include, but are not limited to, azidoproline, aminoproline, mercaptoproline, prolinecarboxylic acid, hydroxyproline, or enantiomers thereof, wherein the enantiomers include (4R)-azidoproline, (4R)-aminoproline, (4R)-mercaptoproline, (4R)-prolinecarboxylic acid, (4R)-hydroxyproline, (4S)-azidoproline, (4S)-aminoproline, (4S)-mercaptoproline, (4S)-prolinecarboxylic acid, and (4S)-hydroxyproline. In one embodiment of the method, the non-saccharide molecules are as defined above. In another embodiment of the method, the polyproline backbone has a formula as defined above. In another embodiment, the method comprises attaching the non-saccharide molecules at pre-determined positions along the polyproline backbone. In yet another embodiment, the method comprises attaching the non-saccharide molecules at equal distances from each other along the polyproline backbone. In one embodiment of the method, the non-saccharide molecules may be attached along more than one different faces of the polyproline backbone. For example, the non-saccharide molecules may be attached along three faces and project from the polyproline backbone. Alternatively, the non-saccharide molecules may be attached along the same face of the polyproline backbone. In one embodiment of the method, the non-saccharide glycosaminoglycan mimetic molecule comprises more than one non-saccharide molecules.

In an eleventh aspect, there is provided a method of promoting neuritogenesis in a patient, comprising administering a non-saccharide glycosaminoglycan mimetic molecule as described above. There is also provided a use of the non-saccharide glycosaminoglycan mimetic molecule as defined above in the manufacture of a medicament for promoting neuritogenesis in a patient. Further provided are the non-saccharide glycosaminoglycan mimetic molecules as defined above for use in promoting neuritogenesis in a patient. In one embodiment, the patient is one who is suffering from a disease selected from the group consisting of a neurodegenerative disease, a viral infection and a malaria infection. Exemplary neurodegenerative diseases in which a non-saccharide glycosaminoglycan mimetic molecule as defined above may be useful include, but are not limited to, Alzheimer's disease, Parkinson's disease and Huntington's disease or the like.

In a twelfth aspect, there is provided a method of inhibiting extravasation of circulating tumor cell into potential metastatic sites in a patient, comprising administering a non-saccharide glycosaminoglycan mimetic molecule as described above. There is also provided a use of the non-saccharide glycosaminoglycan mimetic molecule as defined above in the manufacture of a medicament for inhibiting extravasation of circulating tumor cell into potential metastatic sites in a patient. Further provided are the non-saccharide glycosaminoglycan mimetic molecules as defined above for use in inhibiting extravasation of circulating tumor cell into potential metastatic sites in a patient. In one embodiment, the patient is one who is suffering from a cancer. Exemplary cancers in which a non-saccharide glycosaminoglycan mimetic molecule as defined above may be useful include, but are not limited to, biliary tract cancer, brain cancer, breast cancer, cervical cancer, choriocarcinoma, colon cancer, endometrial cancer, esophageal cancer, gastric cancer, intraepithelial neoplasms, lymphomas, liver cancer, lung cancer (e.g. small cell and non-small cell), melanoma, neuroblastomas, oral cancer, ovarian cancer, pancreas cancer, prostate cancer, rectal cancer, sarcomas, skin cancer, testicular cancer, thyroid cancer, and renal cancer.

### BRIEF DESCRIPTION OF THE DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows ¹H-NMR spectra of NS.

FIG. 2 shows ¹³C-NMR spectra of NS.

FIG. 3 shows analytical HPLC traces (top) and ESI mass data (bottom) of DPPE-(P_{Z})₁₂.

FIG. 4 shows FT-IR spectra of azidopolyprolines and corresponding GAG mimetic agents.

FIG. 5 shows ¹H-NMR spectra of Biotin-(P_{E})₁₂-NS.

FIG. 6 shows ¹H-NMR spectra of Biotin-(PPP_{E})₁₂-NS.

FIG. 7 shows ¹H-NMR spectra of DPPE-(P_{E})₁₂-NS.

FIG. 8 shows CD spectra of (P_{E})₁₂-NS, and (PPP_{E})₁₂-NS at 25° C.

FIG. 9 shows (A) a schematic of the synthesis of a non-saccharide sulfated mimetic (NS), and (B) the conjugation of DPPE/biotin and click reaction. The conditions are as follows: (i) Zn, CaCl₂, NH₄Cl, THF-H₂O, rt, 50%; (ii) LiAlH₄, THF, rt, 60%; (iii) SO₃.TMA, DMF, rt, quant.; (iv) DIPEA, DMF, rt; (v) PyBOP, HOBt, DIPEA, DMF, rt; (vi) NS, CuI, TBTA, DIPEA, DMSO, rt.

FIG. 10 shows (A) sensorgrams showing the interaction of (a) GDNF and (b) GFRα1 at a range of concentrations 0.2-1.0 nM with immobilized (PPP_{E})₁₂-NS, and (B) sensorgram for (P_{E})₁₂-NS binding at various concentrations (0.2, 0.4, 0.6, 0.8 and 1.0 nM from bottom to top) to (a) GDNF and (b) GFRα1.

FIG. 11 shows (A) proposed binding sites of (P_{E})₁₂-NS to GDNF and GFRα1, and (B) proposed binding sites of (PPP_{E})₁₂-NS to GDNF and GFRα1. Positively charged interacting residues are indicated in black.

FIG. 12 shows (A) proposed mechanism of effect for DPPE-(P_{E})₁₂-NS on enhancing GDNF recruitment and for inhibition by (P_{E})12-NS, (B) verification of cell-surface functionalization with a DPPE-Rhodamine analog (scale bar=50.0 μm), (C) and (D) DPPE-(P_{E})₁₂-NS treated PC12 cells show a marked increase in percentage of neurite-bearing cells and neurite length. Complete inhibition was observed with (P_{E})₁₂-NS added to the media. No significant effect was observed when NS was added exogenously.

FIG. 13 shows sensorgrams showing the binding of (A) P-selectin, (B) L-selectin, and (C) E-selectin at various concentrations (5 to 50 nM from bottom to top) with immobilized (PPP_{E})₁₂-NS, and (P_{E})₁₂-NS.

FIG. 14 shows the proposed binding sites of (A) (P_{E})₁₂-NS to P-selectin, and (B) (PPP_{E})₁₂-NS to P-selectin.

FIG. 15 shows the relative activity of (A) Factor Xa, and (B) Factor IIa in the presence of (P_{E})₁₂-NS or heparin.

FIG. 16 shows (A) fluorescence microimages of calcein-labeled B16F10 murine melanoma cells bound to P-selectin-coated surfaces in the presence of heparin or (P_{E})₁₂-NS, and (B) the normalized data representing the percentage of calcein-labeled B16F10 murine melanoma cells bound to P-selectin-coated surfaces, in the presence of heparin or (P_{E})₁₂-NS.

### DETAILED DESCRIPTION OF THE DRAWINGS

### Examples

Non-limiting examples of the invention, including the best mode, and a comparative example will be further described in greater detail by reference to specific Examples, which should not be construed as in any way limiting the scope of the invention.

### Example 1—General Methods

Unless otherwise stated, reactions were performed using anhydrous solvents and in flame-dried glassware under argon atmosphere. All commercial reagents were used as received unless otherwise stated. 1,2-Dipalmitoyl-sn-glycero-3-phosphoethanolamine-N-(succinyl) (sodium salt) (catalogue number 870225P) was purchased from Avanti Polar Lipids, Inc. Thin layer chromatography (TLC) was performed using E. Merck silica gel 60 F254 precoated plates (0.25 mm) and visualization of the developed chromatogram was performed by UV, cerium ammonium molybdate, or ninhydrin stain as necessary. Merck silica gel 60 (particle size 0.040-0.063 mm) was used for flash chromatography. HPLC peptide purification was conducted on Gilson HPLC GX-271 System, equipped with a reverse phase Kromasil® 100-5-C18 column (21.2×250 mm) or a normal phase XBridge™ Prep HILIC column, 5 m (10×100 mm). Gel filtration chromatography (Sephadex G-15 or LH-20 ultrafine: GE Healthcare) was used for the purification of glycosaminoglycan (GAG) mimetic agents.

¹H NMR spectra were recorded on a Bruker AVIII 400 (400 MHz) spectrometer and are reported in parts per million (6) relative to D₂O (4.79 ppm) or (CD₃)₂SO (3.33 ppm). ¹H NMR spectra are reported as follows: chemical shift (6 ppm), multiplicity (s=singlet, br s=broad singlet, t=triplet, m=multiplet), coupling constant in Hz, and integration. ¹³C NMR spectra were obtained on a Bruker AVIII 400 (100 MHz) spectrometer and are reported in terms of chemical shift. Mass spectra were obtained from Waters SQD Quadrupole Mass Spectrometer equipped with Waters Acquity™ Ultra Performance Liquid Chromatography and were recorded in m/z. FTIR spectra were collected using Perkin-Elmer Spectrum 100 and are reported in terms of wavelength (cm⁻¹).

### Abbreviations

SO₃.TMA Sulfur trioxide trimethylamine complex
Ac₂O Acetic anhydride

### DIPEA N,N-Diisopropylethylamine

PEG Polyethylene glycol
TBTU N,N,N′,N′-Tetramethyl-O-(benzotriazol-1-yl)uranium tetrafluoroborate
PyB OP Benzotriazol-1-yl-oxytripyrrolidinophosphonium hexafluorophosphate

### HOBt Hydroxybenzotriazole

TFA Trifluoroacetic acid
DPPE-succinyl-OH sodium salt 1,2-dipalmitoyl-sn-glycero-3-phosphoethanolamine-N-(succinyl) (sodium salt)

### Experimental Procedures

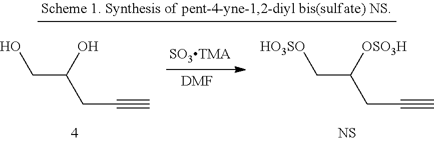

Procedure for the Synthesis of pent-4-yne-1,2-diyl bis(sulfate) (NS)

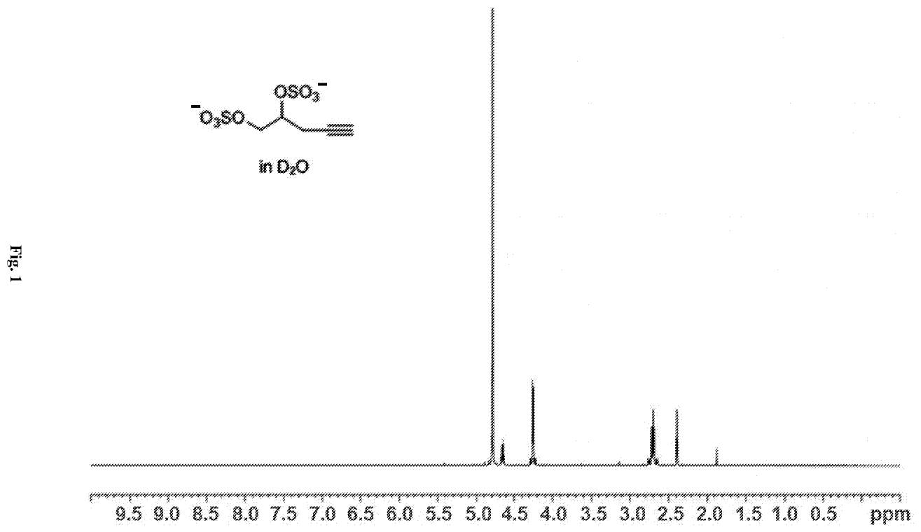

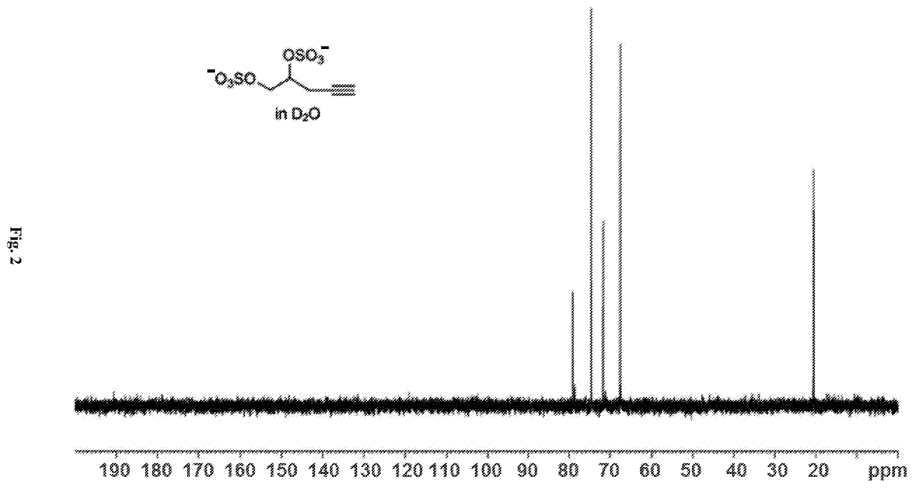

To a solution of pent-4-yne-1,2-diol 4 (100 mg, 1.0 mmol) in DMF (5.8 mL) was added SO₃.TMA (804 mg, 6.0 mmol). The mixture was stirred at 50° C. overnight under argon atmosphere. The product was purified by size exclusion chromatography (Sephadex LH-20 with MeOH/CH₂Cl₂ (1:1)), followed by silica gel chromatography (5%→50% MeOH:CH₂Cl₂). Pent-4-yne-1,2-diyl bis(sulfate) NS was obtained as a white solid (257 mg, 99%, protonated form). ¹H NMR (400 MHz, D₂O): δ 4.68-4.63 (m, 1H), 4.29-4.22 (m, 2H), 2.77-2.64 (m, 2H), 2.39 (t, J=2.7 Hz, 1H) (FIG. 1). ¹³C NMR (100 MHz, D₂O): 79.1, 74.6, 71.7, 67.6, 20.5 (FIG. 2).

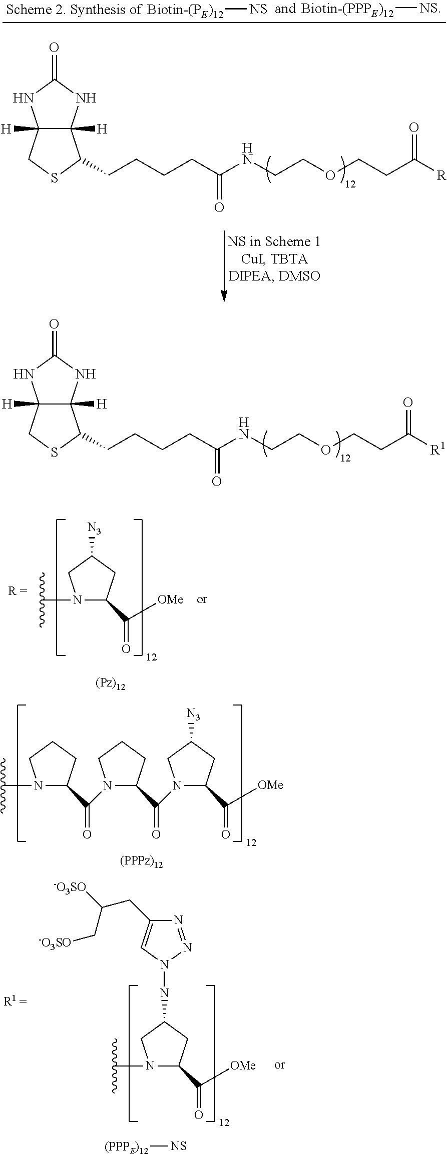

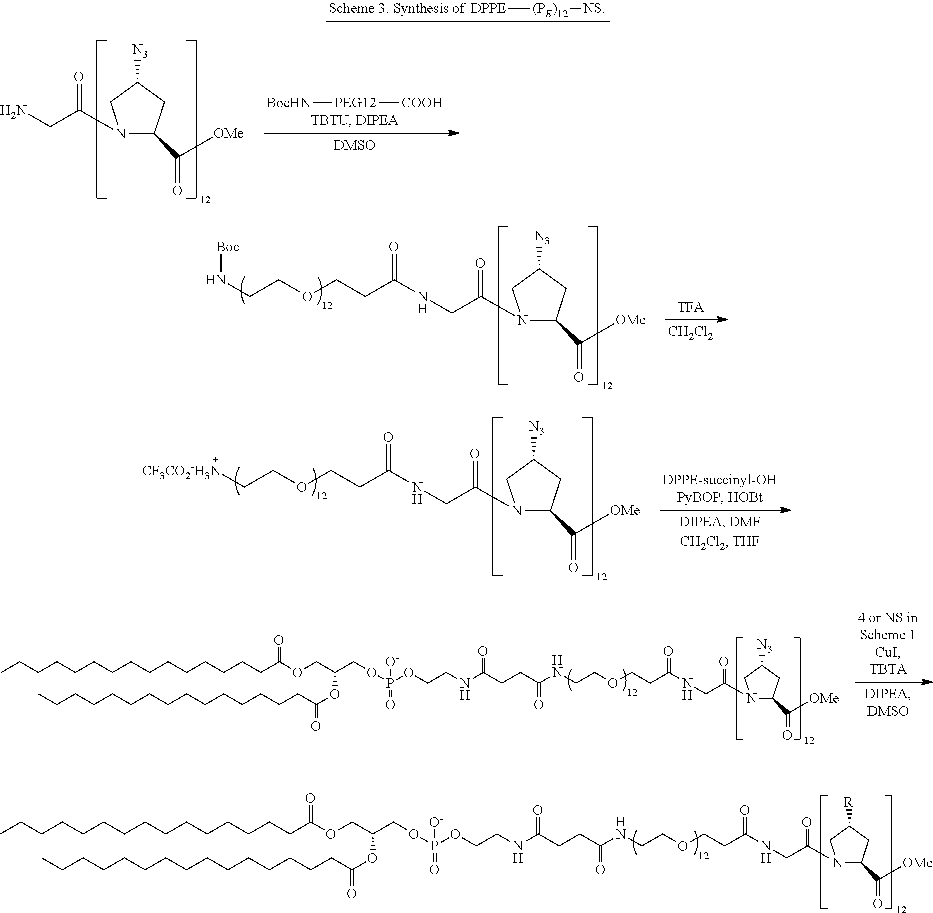

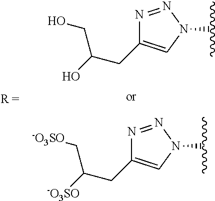

### Peptide Synthesis

Polypeptides ((Pz)₁₂ or (PPPz)₁₂, Biotin-(Pz)₁₂, and Biotin-(PPPz)₁₂) were synthesized as reported previously.^{[10}]

### Procedure for the Synthesis of DPPE-(Pz)₁₂

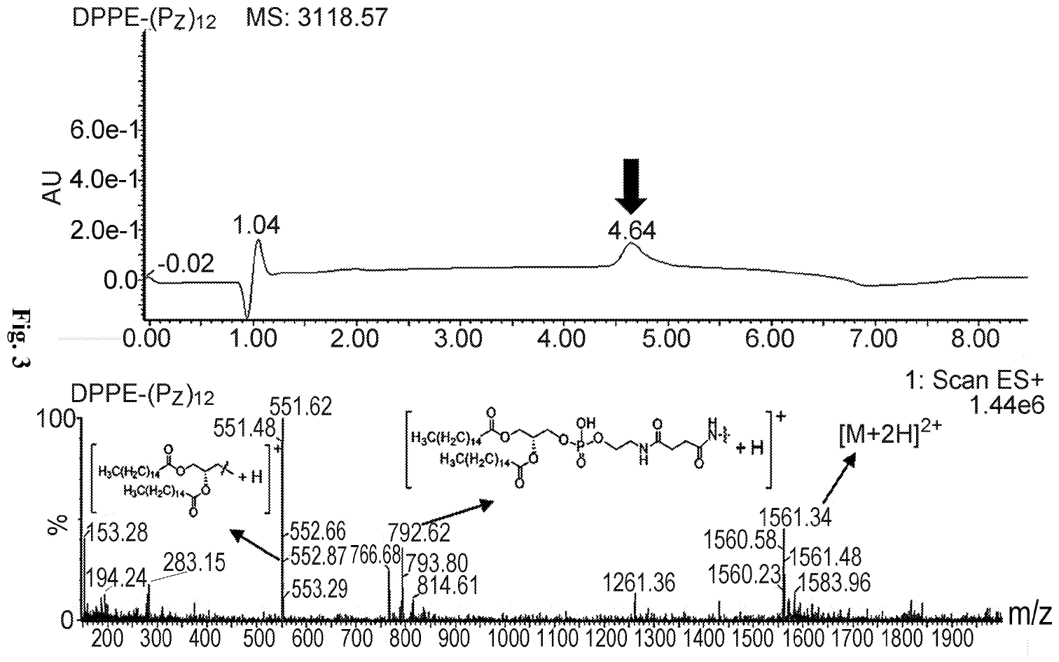

To a solution of H₂N-PEG12-(Pz)₁₂ (1.0 equiv.) in anhydrous DMF/CH₂Cl₂/THF (2:1:1, 0.1 M of final concentration) were added DPPE-succinyl-OH sodium salt (1.0 equiv.), PyBOP (2.0 equiv.), HOBt (2.0 equiv.), and DIPEA (5.0 equiv.). The reaction mixture was stirred at room temperature for 3 days under argon atmosphere. Upon completion, the solvent was removed in vacuo to afford yellow sticky oil. Purification of this oil was carried out by precipitating in acetonitrile (5 mL×2) and methanol (5 mL×2) to afford DPPE-(Pz)₁₂ as a white solid (all excess reagents and impurities were successfully removed). The analytical HPLC traces and ESI mass data of DPPE-(Pz)₁₂ is shown in FIG. 3.

TABLE 1
Analytical HPLC conditions.

| Polyproline | HPLC Condition* | Column** | Retention Time |
|---|---|---|---|
| DPPE-(P_{Z})₁₂ | 1) 25-100% A over 5.5 min | HILIC | 4.64 min |
| | 2) 100% over 2.0 min | | |

*A: CH₃CN (0.1% TFA), B: H₂O (0.1% TFA), % A + % B = 100%, Flow rate = 0.3 mL/min.
**Column: Waters Acquity UPLC ® BEH HILIC 1.7 μm (2.1 × 100 mm)

TABLE 2
ESI-MS.

| Polyproline | MS, calculated | ESI-MS, observed |
|---|---|---|
| DPPE-(P_{Z})₁₂ | 3118.57 | 1561.34 ([M + 2H]²⁺) |

### General Procedure for the Click Reaction

Pent-4-yne-1,2-diyl bis(sulfate) NS (15.6 equiv.), polypeptides containing Pz units (1.0 equiv.), and TBTA (0.3 equiv. per azide) were added into a vial. Under argon atmosphere, the mixture was dissolved in anhydrous DMSO (final concentration: 0.1 M) and copper (I) iodide stock solution in DMSO (0.3 mol % per azide) and DIPEA (48.0 equiv.) were sequentially added. The reaction mixture was then stirred for 14 days at room temperature under argon atmosphere. After complete consumption of the polypeptides, the solvent was removed with continuous nitrogen flow. The resulting mixture was dissolved in 200 μL of 4M aq. NaCl and purified by Sephadex G-15 column (100% H₂O). Upon lyophilisation, the desired GAG mimetic agents were afforded as white solids.

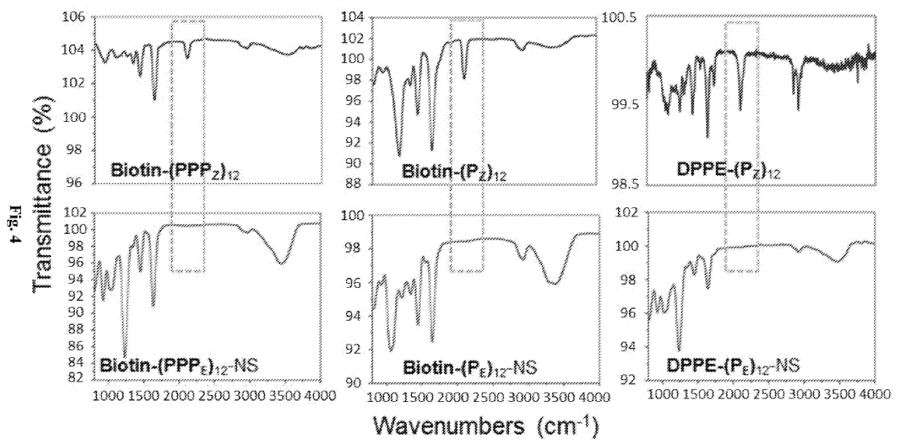

The azide vibrational band (˜2100 cm⁻¹) in FTIR spectra was used to monitor the completion of the click reaction. FTIR was conducted using a Perkin Elmer FTIR Spectrum 100 between 4000 and 800 cm⁻¹ at a spectral resolution of 4 cm⁻¹, with 4 scans per sample. Preparation of the FTIR was done by placing the samples on a germanium stage. The samples were then pressed before the measurement. The disappearance of the azide vibrational band in the spectra of the non-carbohydrate GAG mimetic agents indicated the completion of the coupling reactions (FIG. 4).

### NMR Characterization

Biotin-(P_{E})₁₂—NS

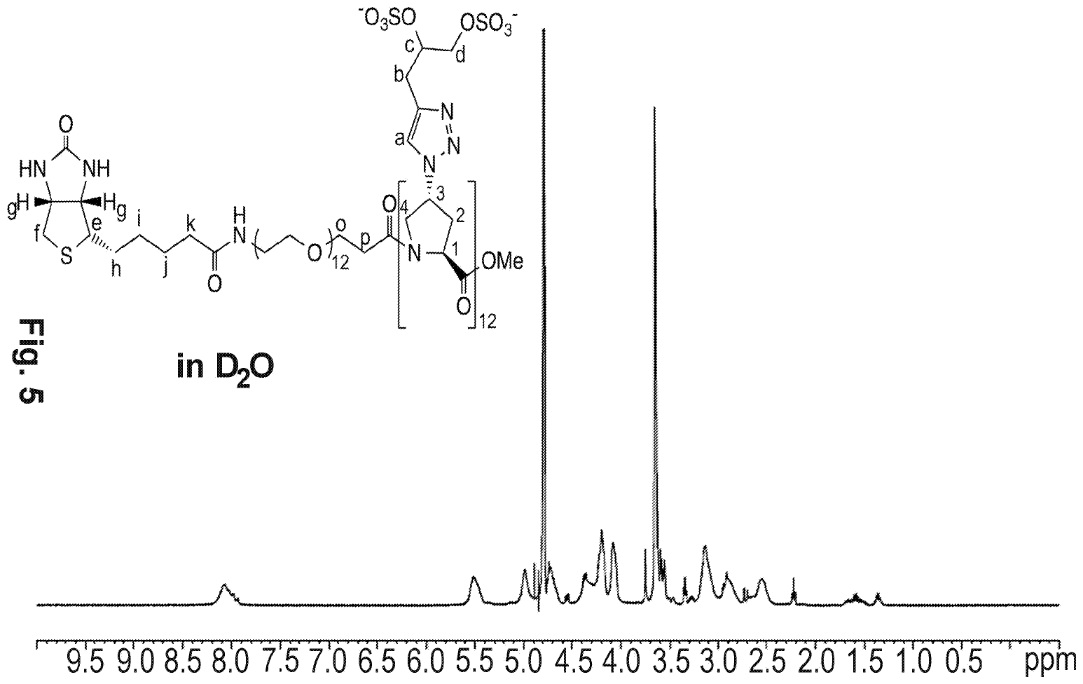

¹H NMR (400 MHz, D₂O): δ 8.24-7.88 (m, 12H, H-a), 5.61-5.39 (m, 12H, H-3), 5.09-4.90 (br s, 12H, H-1), 4.77-4.62 (m, 12H, H-c), 4.59-4.52 (m, 2H, H-g), 4.46-4.14 (m, 36H, H-4, H-d), 4.14-3.99 (m, 12H, H-4′), 3.75 (s, 3H, CO₂CH₃), 3.69-3.53 (m, 48H, OCH₂CH₂O), 3.37-3.24 (m, 3H, H-e, H-o), 3.14 (br s, 24H, H-b), 2.99-2.76 (m, 14H, H-2, H-f), 2.76-2.45 (m, 14H, H-2′, H-p), 2.23 (t, 2H, J=6.0 Hz, H-h), 1.75-1.28 (m, 6H, H-i, H-j, H-k) (FIG. 5).

Biotin-(PPP_{E})₁₂-NS

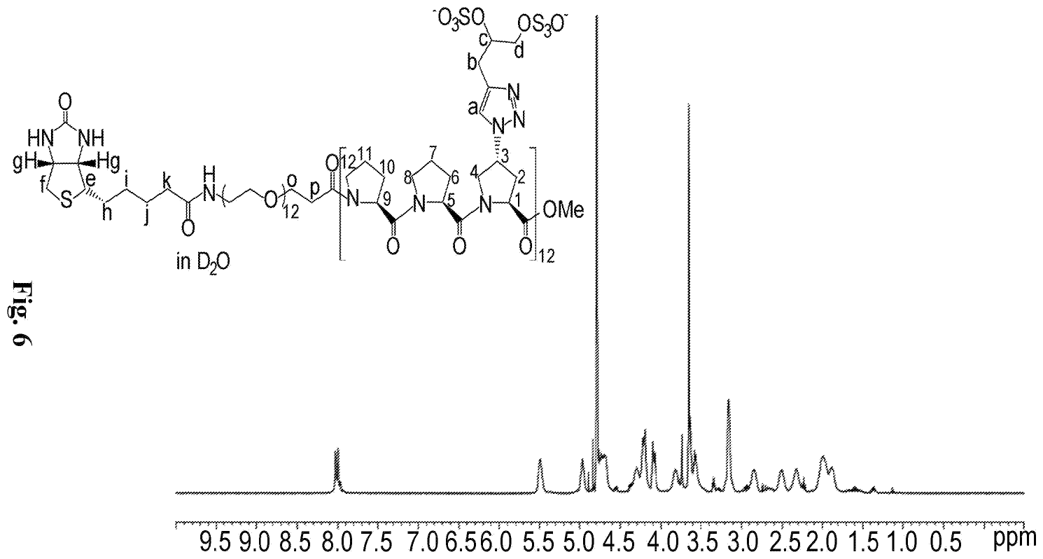

¹H NMR (400 MHz, D₂O): δ 8.06-7.94 (m, 12H, H-a), 5.49 (br s, 12H, H-3), 5.01-4.92 (m, 12H, H-1), 4.76-4.61 (m, 12H, H-c), 4.59-4.50 (m, 2H, H-g), 4.39-3.76 (m, 75H, H-4, H-5, H-8, H-9, H-d), 3.74 (s, 24H, CO₂CH₃), 3.65 (s, 48H, OCH₂CH₂O), 3.62-3.52 (m, 25H, H-12, H-e), 3.37-3.30 (m, 2H, H-o), 3.23-3.05 (m, 36H, H-2, H-b), 2.99-2.68 (m, 14H, H-2′, H-f), 2.68-2.42 (m, 14H, H-6, H-p), 2.42-2.19 (m, 26H, H-6′, H-10, H-h), 2.07-1.79 (m, 60H, H-7, H-10′, H-11), 1.67-1.08 (m, 6H, H-i, H-j, H-k) (FIG. 6).

DPPE-(P_{E})₂—NS

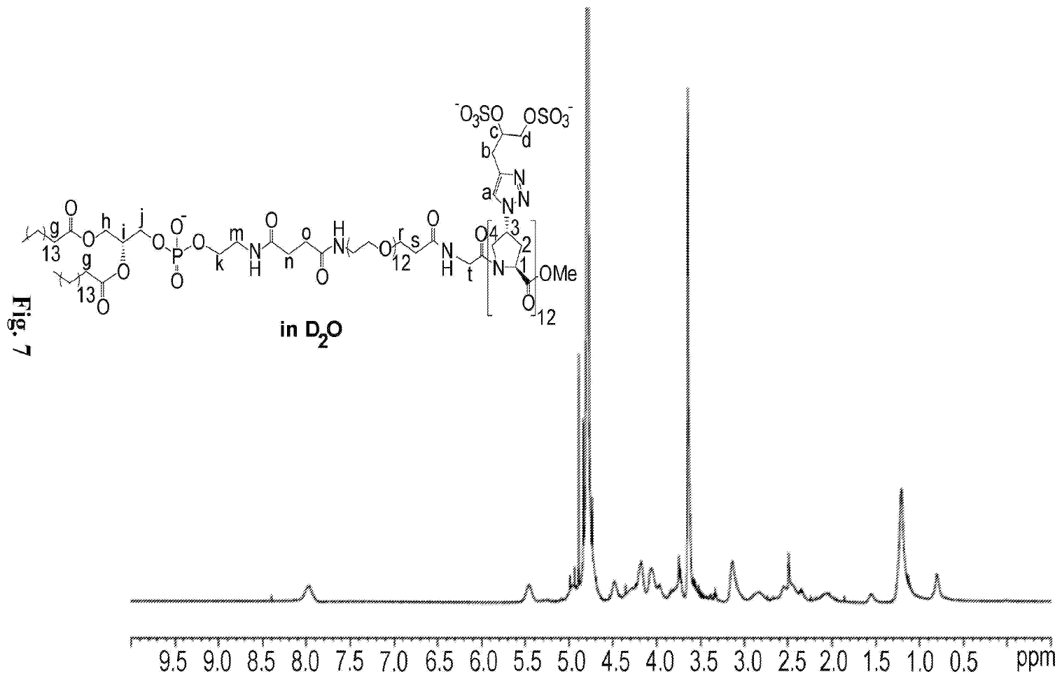

¹H NMR (400 MHz, D₂O): δ 8.10-7.85 (m, 12H, H-a), 5.46 (br s, 12H, H-3), 5.11-4.90 (m, 13H, H-1, H-i), 4.57-4.38 (m, 12H, H-c), 4.38-3.69 (m, 61H, H-4, H-d, H-h, H-j, H-k, H-r, H-t, CO₂CH₃), 3.65 (s, 48H, OCH₂CH₂O), 3.42-3.29 (m, 2H, H-m), 3.14 (br s, 24H, H-b), 2.84 (br s, 12H, H-2), 2.75-2.27 (m, 16H, H-2′, H-o, H-n,), 2.20-1.96 (m, 6H, H-g, H-s), 1.21 (br s, 52H, CH₂CH₃ of DPPE chain), 0.88-0.73 (m, 6H, CH₂CH₃ of DPPE chain) (FIG. 7).

### Circular Dichroism (CD) Analysis

CD spectra were obtained using a Jasco-815 CD spectrometer equipped with a Peltier temperature controller (Jasco PTC-423S/15). 200 μM of sample solutions in 10 mM sodium phosphate-dibasic buffer (pH 7.0) were equilibrated at 4° C. for 24 hr, followed by at room temperature for 1 hr before measurements. Spectra were recorded at 25° C. from 260 to 190 nm. Mean residue ellipticity [0] was calculated as follows;

[θ]=θ/(10·N·c·l)

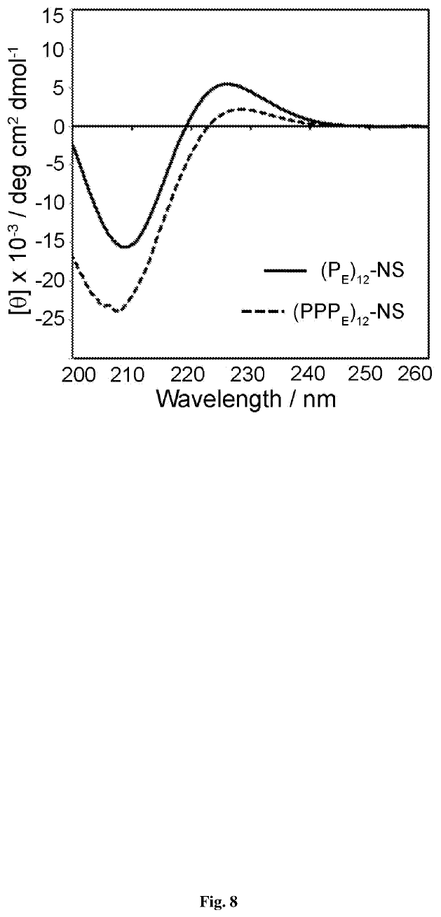

θ represents the ellipticity in millidegrees, N the number of amino acid residues, c the molar concentration in mol L⁻¹, and 1 the cell path length in cm. The CD spectra of (P_{E})₁₂-NS, and (PPP_{E})₁₂-NS at 25° C. is shown in FIG. 8.

### Surface Plasmon Resonance

SPR measurements were performed using a Biacore T100 system (GE Healthcare). The CM5 sensor chip was primed with HBS-EP+ buffer (10 mM HEPES pH 7.4, 150 mM NaCl, 3.4 mM EDTA, 0.005% P20, GE Healthcare) and was activated using the standard amine-coupling protocol (1:1 mixture of 0.4 M EDC and 0.1M NHS). 0.008 mg/ml of streptavidin solution in 10 mM sodium acetate buffer (Acetate 5.0, GE Healthcare) was conjugated and remaining activated groups were quenched with 1 M ethanolamine solution (pH 8.5). The final amount of streptavidin covalently immobilized on the surface was typically 700 RU. Flow cell 1 (or 3) was used as a reference to subtract nonspecific binding, drift, and the bulk refractive index, while flow cell 2 (or 4) was further immobilized with GAG mimetic agents or natural polysaccharides. 5 nM of biotinylated GAG mimetic agents were dissolved in HBS-EP+ buffer and were injected to flow cell 2 (or 4) at 30 μL/min until the baseline response increased by 10, 15 RU, for biotin-(P_{E})₁₂-NS, biotin-(PPP_{E})₁₂-NS, respectively. Immobilized amount is normalized by a molecular weight. For a given affinity measurement, varying amount of protein solutions were successively injected into the flow cells for 240 seconds of contact time and 800 seconds of dissociation time using a flow rate of 50 μL/min at 25° C. Flow cell was regenerated using 2.5 M MgCl₂ at 30 μL/min. Association (kₐ) and dissociation (k_{d}) rate constants were calculated with a 1:1 binding model using Biacore evaluation software, and K_{D} values were calculated from the ratio of k_{d} to kₐ. Kinetic parameters were obtained by fitting curves to a 1:1 Langmuir model with baseline correction.

TABLE 3
Calculated equilibrium binding constants of proteins with
(P_{E})₁₂-NS, (PPP_{E})₁₂-NS.

| | k_{α} (M⁻¹S⁻¹) | k_{d} (S⁻¹) | K_{D} (M)^{a} |
|---|---|---|---|
| GDNF | | | |
| (P_{E})₁₂-NS | 6.42 (± 0.28) × 10⁵ | 2.12 (± 0.07) × 10⁻³ | 3.30 × 10⁻⁹ |
| (PPP_{E})₁₂-NS | ND^{b} | ND^{b} | ND^{b} |
| GFRα1 | | | |
| (P_{E})₁₂-NS | 4.26 (± 0.90) × 10⁵ | 4.9 (± 1.7) × 10⁻² | 1.16 × 10⁻⁷ |
| (PPP_{E} )₁₂-NS | ND^{b} | ND^{b} | ND^{b} |

^{a}K_{D} values were obtained independently from each k_{α} and k_{d} value using Biacore T100 evaluation software v 2.0.4.
^{b}ND = not determined. The exact kinetic parameters were not calculated due to a decrease in response after reaching equilibrium in the association phase.

### Molecular Modeling

The GDNF structure was obtained from the known crystal of GDNF/GFRα1 complex (PDB code: 2V5E)^{[11]} downloaded from the RCSB Protein Data Bank (www.pdb.org). The sulfated pendant units were built using Maestro 9.6 (www.schrodinger.com) and a short polyproline chain was constructed based on previously reported structure.^{[12]} The chain was then elongated by linking multiple copies of the fragment. Sulfated pendant units were included at the respective locations along the polyproline chain. Geometry of the chain was fixed and the side chain conformation of the sulfated pendant units was energy minimized using OPLS_2005 to achieve individual rms deviation of less than 0.1 Å.

The binding domain was determined by first loading the GDNF/GFRα1 complex and removing the water molecules for simplicity. Initial binding sites were first identified by examining the positively charged residues on GDNF. Docking of the GAG mimetic agents was then performed manually. The GAG mimetics were brought to close proximity with the positively charged residues such that the distance between the sulfated pendant units and the positively charged residues were within 3 Å. Steric clashes were avoided during this process. Bonds in the sulfated pendant units and interacting residues on GDNF were rotated to increase potential interactions and the energy was minimized as mentioned above. Potential hydrogen bonding residues were then identified on the binding domains of GDNF. Residues in close proximity of the sulfated pendant units that can participate in hydrogen bonding (Ser, Gln, Asn, Thr, Tyr) were highlighted. The side groups of these residues and the sulfated pendant units were rotated to achieve optimal hydrogen bonding distances of 2-4 Å and bond angles of approximately 150 to 180°. If these manipulations are unsuccessful, hydrogen bonding between the highlighted residue and the sulfated, non-carbohydrate pendant units would be deemed implausible and any structural changes would be undone. Large manipulations to sulfated pendant units and all participating residues were avoided as much as possible to prevent potential disruptions to identified electrostatic interactions. Energies of the participating residues and sulfated pendant units were then minimized.

### Cellular Assays

### General Cell Culture

PC12 cells were maintained in T75 tissue culture flasks in RPMI 1640 medium (ATCC® 30-2001) supplemented with 10% heat-inactivated horse serum (HI-HS, Gibco 26050-088), 5% fetal bovine serum (FBS, Gibco 26170-043) and 1% penicillin/streptomycin (Gibco 15140-122). Stock cultures from liquid nitrogen were grown at 37° C. with 5% CO₂ in a humidified chamber for a minimum of 72 hr before experiments. Cells were seeded on 13 mm round glass coverslips (Paul Marienfeld 0111530). The coverslips were pretreated with 65% nitric acid for three days, washed with distilled water, 70% ethanol and 100% ethanol thrice each for 30 minutes with gentle rocking and then dried in a cell culture hood overnight under UV. For cell attachment, the treated coverslips were coated with laminin (25 μg/mL in PBS; Sigma L2020) at 37° C. for 1 hr, washed thrice with PBS, and then placed at the bottom of a 24-well plate for cell seeding. For all experiments, cells were cultured in RPMI 1640 differentiation media containing 1% HI-HS.

### Neurite Outgrowth Assay

PC12 cells were first harvested and incubated in differentiation media or differentiation media supplemented with 30 μM of DPPE-(P_{E})₁₂-NS for 2 h. After incubation, the cells were centrifuged and rinsed twice with fresh differentiation medium and then seeded onto laminin-coated coverslips at a density of 100 cells/mm². After allowing the cells to attach for 1 hr at 37° C., the medium was replaced with fresh differentiation medium supplemented with 200 ng/mL GDNF and 1 μg/mL GFRα1. As additional controls, (P_{E})₁₂-NS or NS were further added to the supplemented medium to a final concentration of 20 μM for untreated cells. After 72 h, the cells were fixed with 4% paraformaldehyde solution (Tokyo Chemical Industry 30525-89-4 in PBS) for 15 minutes at room temperature and then rinsed with PBS. Bright-field images were taken using an Olympus IX71 Inverted Microscope at 20× magnification under phase contrast. For each condition, 400-500 randomly selected single cells were counted. The percentage of neurite-bearing cells was determined by counting the number of cells with neurites longer than the cell body. Experiments were repeated three times and done in duplicate each time.

### Detecting Cell Surface Functionalization

PC12 cells were harvested and functionalized with DPPE-Rhodamine analog of DPPE-(P_{E})₁₂-NS as described above. After incubation, the cells were centrifuged and rinsed with fresh differentiation medium.

The cells were then incubated for 30 min in Hoechst dye (1:300 dilution in PBS/4% FBS) and rinsed again in PBS/4% FBS. The cells were re-suspended in PBS/4% FBS and fluorescent images were taken by mounting the cell suspension on a glass coverslip and imaging with an Olympus IX71 Inverted Microscope at 20× magnification.

### Example 2—Designing a New Class of Non-Saccharide Glycosaminoglycan Mimetic Molecules

Previously reported polyproline-based glycomimetic strategy was used as the basis for developing a new class of non-saccharide glycosaminoglycan mimetic molecules due to the ability to control the spatial display of bioactive epitopes on the rigid, well defined polyproline type II (PPII) helical backbone^{[13-14}]. The non-saccharide epitope (NS) was designed to contain a primary and secondary sulfation group and this epitope was incorporated onto the PPII helix by click reaction. Further, a minimal distance was maintained between the peptide backbone and the sulfation groups for maximal positional control. A biotin- or DPPE-conjugated PEG₁₂ chain was introduced to facilitate surface attachment or cell membrane insertion, respectively.

Two non-saccharide mimetics were hence designed based on the above considerations: (i) a distributed design ((P_{E})₁₂-NS) with negatively charged NS on all three faces of the helix and (ii) a single-facial design ((PPP_{E})₁₂-NS) with NS on only one face of the helix. These non-saccharide mimetics contain the same number of NS moieties, differing only in their spatial display.

### Example 3—Synthesis of NS, Conjugation of DPPE/Biotin and Click Reaction

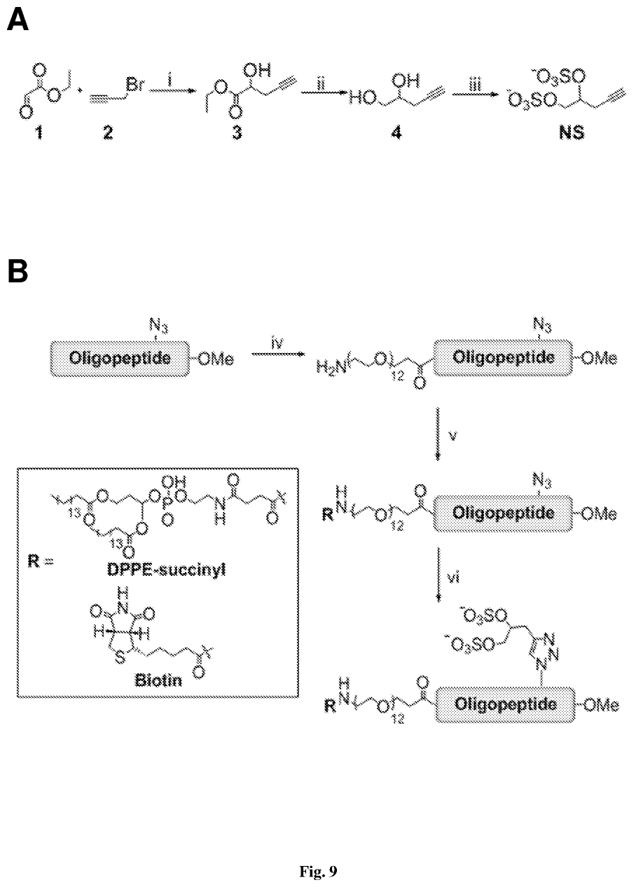

The molecular synthesis of the glycosaminoglycan mimetics was then carried out based on the designs described above. The synthesis was conducted as illustrated in FIG. 9. Firstly, a zinc Barbier reaction between ethyl glyoxylate and propargyl bromide afforded the alkyne ester 3.^{[15]} This step was performed with Zn, CaCl₂, NH₄Cl, and THF-H₂O at room temperature in 50% yield. Subsequent reduction of 3 by lithium aluminum hydride yielded the diol 4.^{[16]} This step was performed with LiAlH₄, and THF at room temperature in 60% yield. The desired non-saccharide sulfated mimetic (NS) was then delivered by sulfation of 4 with SO₃.trimethylamine complex. This step was performed with SO₃.TMA, and DMF at room temperature, in 99% yield. The polyproline scaffolds ((P_{E})₁₂ and (PPP_{E})₁₂) were prepared by standard Boc chemistry in solution phase. PEG₁₂ was conjugated onto the polyproline scaffolds using DIPEA base (in the presence of DIPEA, and DMF at room temperature), while DPPE succinyl or biotin were introduced into the polyproline-PEG scaffolds by amide coupling (in the presence of DIPEA, and DMF at room temperature). The non-saccharide sulfated mimetics ((P_{E})₁₂-NS and (PPP_{E})₁₂-NS) were then prepared by conjugating NS to P_{AZ} residues on the scaffolds via click reaction in the presence of copper (I) iodide (in the presence of CuI, TBTA, DIPEA, and DMSO at room temperature) (FIG. 9B). The reaction was monitored by FT-IR spectra and 1H NMR spectra, with the completion of the coupling reactions marked by disappearance of the azide vibrational band at 2100 cm⁻¹ in FT-IR and the appearance of characteristic peaks from the 1,2,3-triazole linkage in the 1H NMR spectra

### Example 4—Non-Saccharide Glycosaminoglycan as Biological Mimetics of Cell-Surface Heparan Sulfate on PC12 Cells by Regulating the GDNF/GFRα1 Recruitment Process and Leading to Enhanced Neuritogenesis

### Surface Plasmon Resonance (SPR) to Quantify and Analyze Non-Saccharide Glycosaminoglycan Molecule Binding to GDNF and GFRα1

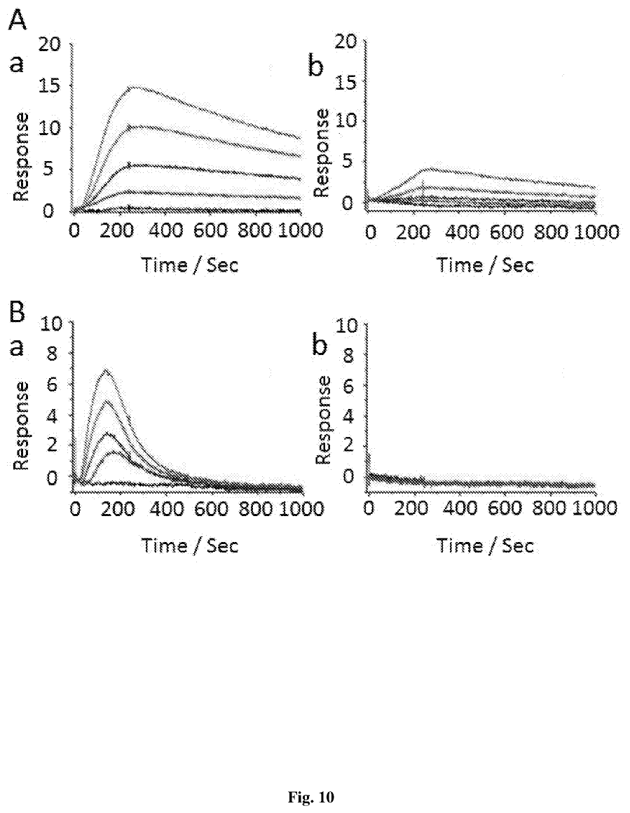

With the non-saccharide glycosaminoglycan mimetics in hand, SPR was employed to facilitate quantitative, real-time kinetic analysis of non-saccharide glycosaminoglycan mimetic binding to GDNF and GFRα1. Biotinylated (P_{E})₁₂-NS and (PPP_{E})₁₂-NS were immobilized on the surface of an streptavidin-coated CM sensor chip and their binding affinities with GDNF and GFRα1 were investigated. Surprisingly, the sensorgrams showed that although both non-saccharide mimetics recognize GDNF and GFRα1 in a dose-dependent manner, (P_{E})₁₂-NS demonstrated a far higher binding affinity to both proteins than (PPP_{E})₁₂-NS (FIG. 10). This result stands in direct contrast to previously reported glycopeptide study with NGF, where the single-facial design was shown to elicit the highest response^{[6]}.

### Computational Modelling to Elucidate the Mechanism of Non-Saccharide Glycosaminoglycan Molecule Binding to GDNF and GFRα1

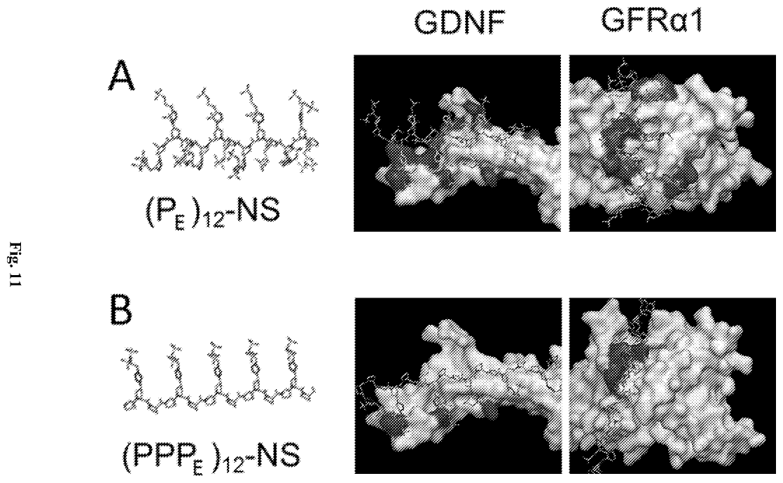

Computational modeling was used to elucidate the mechanism behind this surprising finding described above. Models of GDNF and GFRα1 were retrieved from the protein database (PDB no: 2v5e) and regions on both proteins containing basic residues were highlighted and identified as probable interaction sites. The predicted binding site on the surface of GDNF contains a cluster of 8 basic residues within a range of 9 Å, offering a highly charged group of binding sites for our non-saccharide glycosaminoglycan mimetic. Similarly, a docking site containing 9 basic residues was identified for GFRα1. The model of (P_{E})₁₂-NS indicates that the 12 pendant sulfate groups are arranged in three parallel lines spaced 10 Å apart. Separately docking this model to the binding sites of GDNF and GFRα1 demonstrated that 9 basic residues on GDNF are able to interact simultaneously with 7 pendant sulfate groups of (P_{E})₁₂-NS. Similarly, 9 basic residues on GFRα1 interact with 8 pendant sulfate groups of (P_{E})₁₂-NS (FIG. 11). Importantly, it was observed that NS moieties on two faces on the helix acted as binding residues in both docking studies. In contrast, (PPP_{E})₁₂-NS consists of a single linear array of sulfate groups. Docking of (PPP_{E})₁₂-NS to GDNF and GFRα1 indicated the presence of 4 and 2 interacting residues respectively. The lack of binding residues on multiple faces of the helix in (PPP_{E})₁₂-NS significantly reduces the number of interacting residues in GDNF and GFRα1, explaining the weaker overall binding affinity observed between the proteins and (PPP_{E})₁₂-NS. Taken together, the SPR and modeling results show the suitability of the non-saccharide glycosaminoglycan mimetics to efficiently recruit target proteins and highlight the ability to modulate protein binding affinity by precisely controlling the spatial positioning of non-saccharide bioactive residues.

Detection of Passive Exogenous Insertion of Non-Saccharide Glycosaminoglycan Molecules onto Cell Surface

After demonstrating the effectiveness of the non-saccharide glycosaminoglycan mimetic in the recruitment of target proteins, their efficacy in a biological milieu was tested. GDNF mediated signaling of neuronal cells is known to require cell-surface heparin sulphate (HS) proteoglycans as well as the known components of its receptor complex, c-Ret and GFRα1.^{[17]} Cell-surface HS plays a crucial role in recruitment of GDNF and plays a critical role in c-Ret phosphorylation,^{[17a]} leading to the activation of multiple intracellular signal transduction processes. The GDNF pathway is highly important in the development and maintenance of dopaminergic neurons,^{[17c][18]} marking it as a promising avenue for the treatment for Parkinson's disease.^{[1}9]

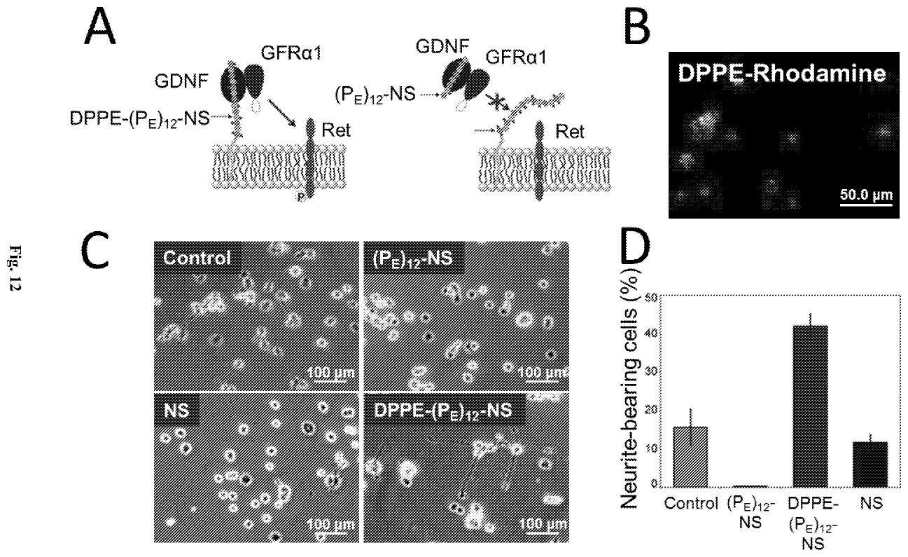

Cell surface HS was mimicked with the non-saccharide glycosaminoglycan mimetics by introducing them onto the cell surface. For this purpose, (P_{E})₁₂-NS was conjugated to DPPE (DPPE-(P_{E})₁₂-NS) in order to allow for passive exogenous insertion of the mimetic into the cell membrane.^{[20]} This method has the benefit of not perturbing any cellular activity, allowing isolation of the effects of the glycosaminoglycan mimetics. The successful incorporation of DPPE-functionalized materials onto the cell surface was verified via fluorescent microscopy with a rhodamine-functionalized analog (FIG. 12B).

### Biological Efficacy of Non-Saccharide Glycosaminoglycan Molecules on GDNF-Mediated Neuritogenesis in Rats

The biological efficacy of (P_{E})₁₂-NS was validated by investigating its effect on the GDNF-mediated neuritogenesis of the rat pheochromocytoma cell line PC12. GDNF triggers neuritogenesis in PC12 cells in a dose-dependent manner,^{[21]} allowing the evaluation of the biological efficacy of DPPE-(P_{E})₁₂-NS to be performed. Notably, as PC12 cells do not express GFRα1, soluble GFRα1 was also added to the culture media to elicit a response of these cells to GDNF. Compared to untreated cells, cells treated with DPPE-(P_{E})₁₂-NS demonstrated dramatically stimulated neurite extension, with the percentage of neurite-bearing cells increasing from 16% in the control to 45% for the treated cells (FIGS. 12C and D), indicating that DPPE-(P_{E})₁₂-NS was able to enhance GDNF/GFRα1 recruitment (FIGS. 12A, C and D). It was further observed that direct addition of NS into the culture media had no discernable effect on neuritogenesis, indicating that the enhancement seen from DPPE-(P_{E})₁₂-NS was not due to sulfotransferases activity on existing cell-surface glycosaminoglycan, but rather due to direct biological action of the non-saccharide glycosaminoglycan mimetic. Moreover, the addition of exogenous biotin-(P_{E})₁₂-NS into the cell culture media completely inhibited neuritogenesis, indicating competitive inhibition between biotin-(P_{E})₁₂-NS and cell surface HS for GDNF/GFRα1 (FIGS. 12A, C and D). These results show the non-saccharide glycosaminoglycan can successfully mimic HS function on GDNF-mediated signaling, and demonstrate the applicability of the strategy in a biological system.

### Example 5—Non-Saccharide Glycosaminoglycan Biological Mimetics as Inhibitors of Cell Adhesion Molecules

### Surface Plasmon Resonance (SPR) to Quantify and Analyze Non-Saccharide Glycosaminoglycan Molecule Binding to Selectins

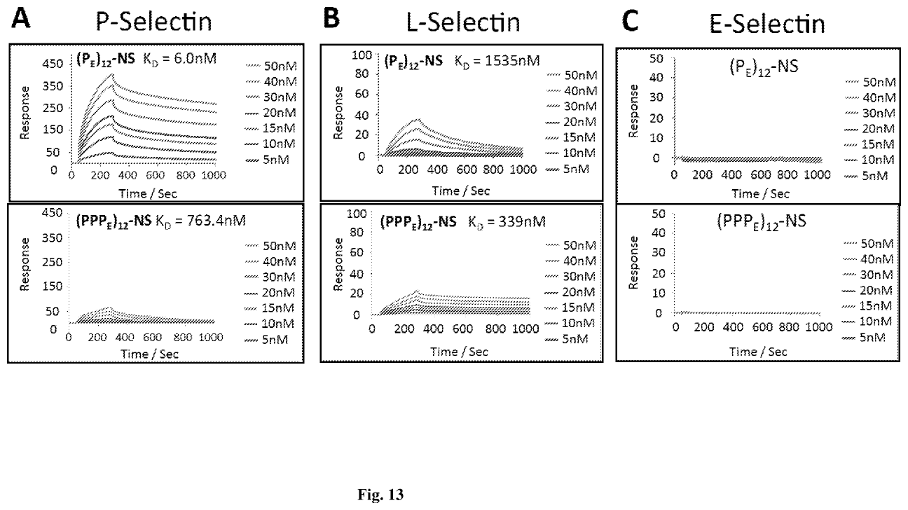

Quantitative, real-time kinetic analysis of the binding of non-saccharide glycosaminoglycan mimetic molecules to selectins was performed using a surface plasmon resonance (Biacore T100, GE Healthcare Life Sciences). Biotinylated glycomimetics were immobilized (to levels normalized according to their molecular weights) onto sensor chips via binding with streptavidin that had been conjugated onto the carboxymethylated dextran matrix using N-Hydroxysuccinimide/ethyl(dimethyl-aminopropyl) carbodiimide chemistry. For binding between glycomimetics to P-selectin (FIG. 13A), the distributed conformation led to a stronger binding affinity as compared to the single facial conformation (i.e. (P_{E})₁₂-NS>(PPP_{E})₁₂-NS). Binding to L-selectin was generally weaker (FIG. 13B). Interestingly for L-selectin, the single facial conformation resulted to significantly slow dissociation rates and therefore, a stronger binding affinity than the distributed conformation (i.e. (PPP_{E})₁₂-NS>(P_{E})₁₂-NS). Similar to heparin and chondroitin sulfate-E, none of the glycomimetics bound to E-selectin, thus maintaining the same specificity as the natural glycosaminoglycan (FIG. 13C).

### Computational Modelling to Elucidate the Mechanism of Non-Saccharide Glycosaminoglycan Molecule Binding to P-Selectin

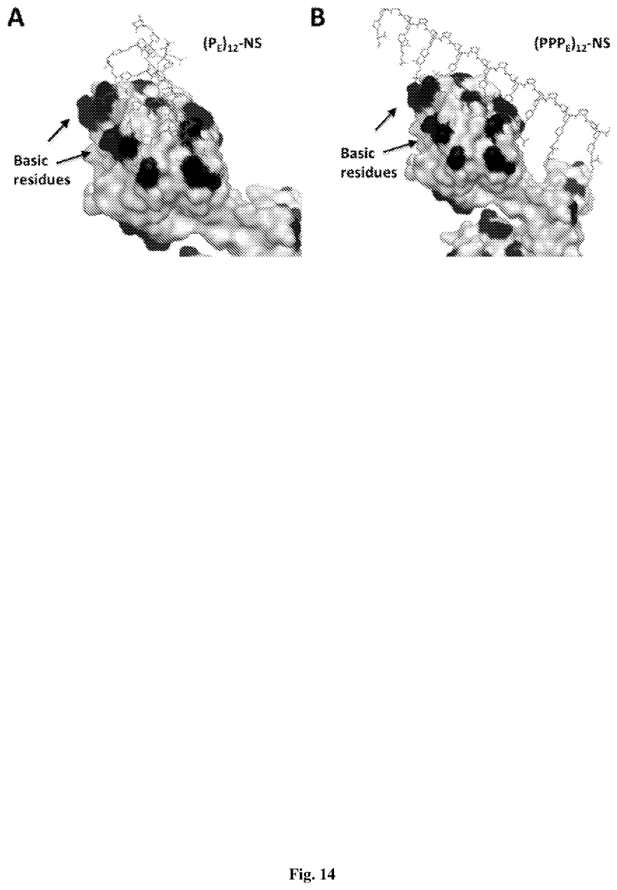

The above data revealed (P_{E})₁₂-NS as a promising candidate for inhibiting P-selectin, since it displayed a binding affinity (K_{D}: 6 nM) far stronger than that observed with heparin (K_{D}: 190.5 nM) and chondroitin sulfate-E (K_{D}: 157.9 nM). Computational modeling was conducted to gain insight into the advantage of the distributed conformation over the single facial conformation. In the protein model of P-selectin (Protein database no. 1G1S), a cluster of 14 basic residues could be found on the surface of the protein and was organized into 3 rows, each spaced approximately 10-15 Å from adjacent ones. On the other hand for (P_{E})₁₂-NS, pendant sulfate groups along each of the 3 rows around the polyproline scaffold were spaced 10 Å apart. Molecular docking of (P_{E})₁₂-NS on P-selectin revealed that 2 rows of pendant sulfate groups on (P_{E})₁₂-NS could line up compatibly against the rows of basic residues on P-selectin, resulting in 10 basic residues interacting closely with 8 pendant sulfate groups (FIG. 14A). On the other hand, (PPP_{E})₁₂-NS only offered a single row of pendant sulfate groups to bring 4 pendant sulfate groups into close interactions with 6 basic residues (FIG. 14B). These modeling studies revealed the molecular basis for the stronger binding between (P_{E})₁₂-NS and P-selectin and lent support to the importance of precisely controlling spatial conformation to position key chemical motifs appropriately over putative binding sites on target proteins.

### Effect of Non-Saccharide Glycosaminoglycan Molecules Versus Heparin on the Activity of Blood Coagulation Factors

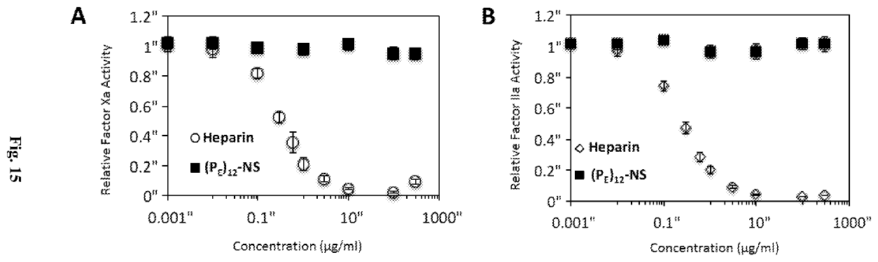

One common drawback hampering the clinical application of negatively charged glycomimetics is the potentially hazardous tendency to activate anti-thrombin, which in turn severely impairs the ability of blood coagulation factors (e.g. Factor Xa and IIa) in the final common pathway to orchestrate blood coagulation. To verify the safety and clinical potential of the engineered non-saccharide glycomimetics of tunable spatial conformation, the effect of (P_{E})₁₂-NS and heparin on the activity of blood coagulation factors, Factor Xa and IIa in the presence of anti-thrombin, was assessed. This assessment was performed using BIOPHEN Heparin Anti-Xa and Anti-IIa assays (Aniara) where the inhibitory effect of anti-thrombin on Factor Xa and IIa was determined by the ability of the factors to catalyze the hydrolysis of factor-specific chromogenic substrates. The resulting data showed that heparin, being a potent anti-coagulant, abolished the activity of Factor Xa and IIa completely at concentrations above 10 ug/ml (FIGS. 15A-B). In contrast, the activity of Factor Xa and IIa remained unaffected in the presence of (P_{E})₁₂-NS at concentrations as high as 300 ug/ml. The use of spatial conformation to present key chemical motifs toward selectins is therefore specific and independent of other proteins that could lead to undesirable consequences if concomitant enhanced binding were to be seen.

### Effect of Non-Saccharide Glycosaminoglycan Molecules Versus Heparin on the Inhibition of P-Selectin-Mediated Adhesion of Tumor Cells

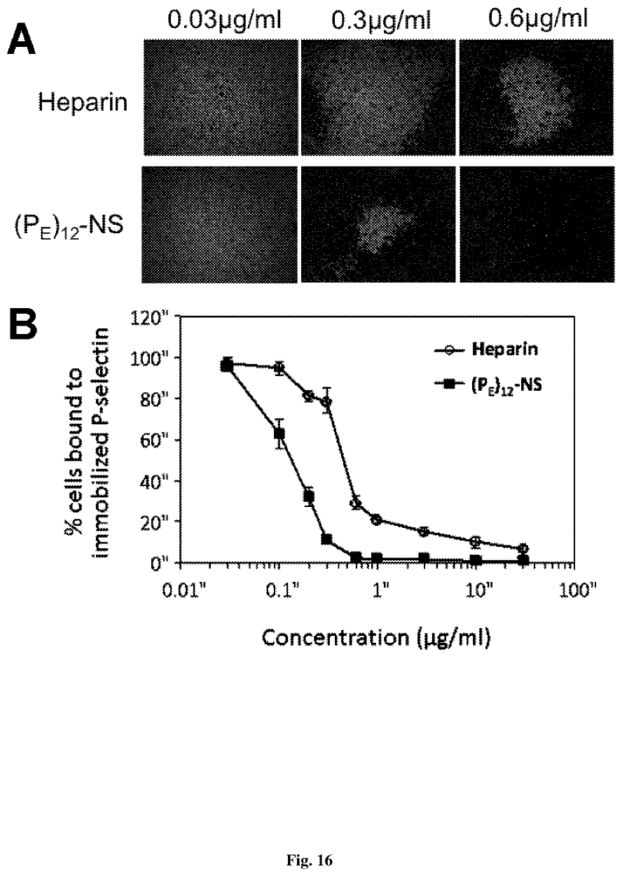

Circulating tumor cells display several moieties such as P-selectin glycoprotein ligand-1 (PSGL-1)²² or chondroitin sulfate glycosaminoglycan²³⁻²⁴ and can tether onto blood vessel wall either directly via P-selectin-expressing endothelial cells or indirectly via the formation of arrested emboli with P-selectin-expressing platelets. Through this manner, P-selectin is instrumental in the extravasation of circulating tumor cells and the commencement of their invasion into potential metastatic sites. Any molecule that can bind to P-selectin can potentially compete with the ligands displayed on tumor cells and thereby inhibit the adhesion of tumor cells to P-selectin. As such, it was examined if the strong binding affinity of (P_{E})₁₂-NS toward P-selectin could translate into inhibition of P-selectin-mediated adhesion of tumor cells. To do so, recombinant P-selectin chimera protein with a Fc region was immobilized on Protein A-coated surfaces. The adhesion of calcein-labeled B16F10 murine melanoma cells to these surfaces in the presence of heparin or (P_{E})₁₂-NS was then visualized (FIG. 16A) and quantified by fluorescence microplate reader (FIG. 16B). In agreement with previous reports, naturally derived heparin was very effective in inhibiting adhesion of B16F10 cells to immobilized P-selectin. Remarkably, (P_{E})₁₂-NS was not only effective but could also outperform heparin in the following aspects. First, IC₅₀ of (P_{E})₁₂-NS, as determined by a non-linear regression fitting model, was significantly lower than heparin (0.18 μg/ml vs. 0.49 μg/ml respectively). Second, (P_{E})₁₂-NS could achieve near-complete inhibition beginning from 0.6 μg/ml (FIG. 16A). This was evidently more efficient than heparin, which could only do so beginning from 30 μg/ml. This is of paramount importance given the goal of therapeutics against cancer metastasis is not simply to reduce the disease burden, but to eliminate as completely as possible any chance of tumor cells anchoring onto blood vessel walls.

### Applications

In summary, a series of non-saccharide glycosaminoglycan mimetics has been developed via the precise spatial positioning of negatively charged sulfation groups on a polyproline backbone. The greatly simplified synthesis of the non-saccharide sulfated moiety compared to carbohydrate-based materials makes this approach highly attractive and practical in the design of glycosaminoglycan mimetics. Variations in the spatial positioning of these charged sulfation units would allow for different protein binding specificities and hence different biological functionalities. These findings highlight the potential of employing spatially-defined non-saccharide multivalent architectures as glycosaminoglycan mimetics. Specifically, the mimetics demonstrate controllable binding affinity to GDNF/GFRα1 through varying epitope display, and are able to mimic natural HS in modulating the neuritogenesis of PC12 cells. Furthermore, glycomimetic-based selectin inhibitors developed based on this method were clinically compelling in terms of both safety and efficacy. The findings are anticipated to provide a useful tool for further development of target-specific therapeutic agents and target-specific inhibitors, exploration of cellular glycosaminoglycan functions and also manipulation of their functions in vivo.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### REFERENCES

(1) Bertozzi, C. R.; Kiessling; L., L. Science 2001, 291, 2357.
(2) (a) Miller, G. J.; Hansen, S. U.; Avizienyte, E.; Rushton, G.; Cole, C.; Jayson, G. C.; Gardiner, J. M. Chemical Science 2013, 4, 3218; (b) Tully, S. E.; Rawat, M.; Hsieh-Wilson, L. C. Journal of the American Chemical Society 2006, 128, 7740; (c) de Paz, J. L.; Noti, C.; Seeberger, P. H. Journal of the American Chemical Society 2006, 128, 2766.
(3) (a) Xiao, C.; Zhao, C.; He, P.; Tang, Z.; Chen, X.; Jing, X. Macromolecular Rapid Communications 2010, 31, 991; (b) Oh, Y. I.; Sheng, G. J.; Chang, S.-K.; Hsieh-Wilson, L. C. Angewandte Chemie International Edition 2013, 52, 11796; (c) Sheng, G. J.; Oh, Y. I.; Chang, S.-K.; Hsieh-Wilson, L. C. Journal of the American Chemical Society 2013, 135, 10898; (d) Rawat, M.; Gama, C. I.; Matson, J. B.; Hsieh-Wilson, L. C. Journal of the American Chemical Society 2008, 130, 2959; (e) Liu, S.; Kiick, K. L. Macromolecules 2008, 41, 764; (f) Takasu, A.; Houjyou, T.; Inai, Y.; Hirabayashi, T. Biomacromolecules 2002, 3, 775.
(4) Al-Horani, R. A.; Karuturi, R.; Verespy, S., 3rd; Desai, U. R. Methods in molecular biology (Clifton, N.J.) 2015, 1229, 49.
(5) (a) Al-Horani, R. A.; Liang, A.; Desai, U. R. Journal of medicinal chemistry 2011, 54, 6125; (b) Gunnarsson, G. T.; Desai, U. R. Journal of medicinal chemistry 2002, 45, 1233.
(6) Mousa, S. A.; Linhardt, R.; Francis, J. L; Amirkhosravi, A. Thrombosis and Haemostasis 2006, 96, 816.
(7) Sudaha T.; Phillips P.; Kanaan C.; Linhardt. R. J.; Borsig L.; Mousa S. A. Clinical and Experimental Metastasis 2012, 29, 431.
(8) Casu, B.; Vlodavsky, I.; Sanderson, R. D. Pathophysiology of Haemostasis and Thrombosis 2009, 36, 195.
(9) Kishimoto, T. K; Viswanathan, K.; Ganguly, T.; Elankumaran, S.; Smith, S.; Pelzer, K.; Lansing, J. C.; Sriranganathan, N.; Zhao, G.; Galcheva-Gargova, Z.; Al-Hakim, A.; Bailey, G. S.; Fraser, B.; Roy, S.; Rogers-Cotrone, T.; Buhse, L.; Whary, M.; Fox, J.; Nasr, M.; Dal Pan, G. J.; Shriver, Z.; Langer, R. S.; Venkataraman, G.; Austen, K. F.; Woodcock, J.; Sasisekharan, R. The New England Journal of Medicine 2008, 358, 2457.
(10) Liu, P.; Chen, L.; Toh, J. K. C.; Ang, Y. L.; Jee, J.-E.; Lim, J.; Lee, S. S.; Lee, S.-G. Chemical Science 2015, 6, 450.
(11) Parkash, V.; Leppanen, V.-M.; Virtanen, H.; Jurvansuu, J. M.; Bespalov, M. M.; Sidorova, Y. A.; Runeberg-Roos, P.; Saarma, M.; Goldman, A. The Journal of Biological Chemistry. 2008, 283, 35164.
(12) Cowan, P. M.; McGavin, S. Nature 1995, 176, 501.
(13) Lee, S-G.; Lee, S-S.; Lim, J.; Cheong, J-L. 2014 WO 2014/175838Δ1.
(14) (a) Nagel, Y. A.; Kuemin, M.; Wennemers, H. Chimia 2011, 65, 264; (b) Nagel, L.; Budke, C.; Erdmann, R. S.; Dreyer, A.; Wennemers, H.; Koop, T.; Sewald, N. Chemistry—A European Journal 2012, 18, 12783; (c) Kuimin, M.; Sonntag, L.-S.; Wennemers, H. Journal of the American Chemical Society 2007, 129, 466; (d) Fillon, Y. A.; Anderson, J. P.; Chmielewski, J. Journal of the American Chemical Society 2005, 127, 11798.
(15) Jiang, X.; Vogel, E. B.; Smith, M. R.; Baker, G. L. Macromolecules 2008, 41, 1937.
(16) Donohoe, T. J.; Ironmonger, A.; Kershaw, N. M. Angewandte Chemie International Edition 2008, 47, 7314.
(17) (a) Barnett, M. W.; Fisher, C. E.; Perona-Wright, G.; Davies, J. A. Journal of cell science 2002, 115, 4495; (b) Davies, J. A.; Yates, E. A.; Turnbull, J. E. Growth Factors 2003, 21, 109; (c) Airaksinen, M. S.; Saarma, M. Nature reviews. Neuroscience 2002, 3, 383; (d) Costantini, F.; Shakya, R. BioEssays: news and reviews in molecular, cellular and developmental biology 2006, 28, 117; (e) Takahashi, M. Cytokine & growth factor reviews 2001, 12, 361.
(18) (a) Bourque, M. J.; Trudeau, L. E. The European journal of neuroscience 2000, 12, 3172; (b) Engele, J.; Franke, B. Cell and tissue research 1996, 286, 235; (c) Lin, L.; Doherty, D.; Lile, J.; Bektesh, S.; Collins, F. Science 1993, 260, 1130.
(19) Kirik, D.; Georgievska, B.; Bjorklund, A. Nature Neuroscience 2004, 7, 105.
(20) (a) Huang, M. L.; Smith, R. A. A.; Trieger, G. W.; Godula, K. Journal of the American Chemical Society 2014, 136, 10565; (b) Rabuka, D.; Forstner, M. B.; Groves, J. T.; Bertozzi, C. R. Journal of the American Chemical Society 2008, 130, 5947; (c) Rabuka, D.; Parthasarathy, R.; Lee, G. S.; Chen, X.; Groves, J. T.; Bertozzi, C. R. Journal of the American Chemical Society 2007, 129, 5462.
(21) (a) Wissel, K.; Stover, T.; Hofmann, N. S.; Chernajovsky, Y.; Daly, G.; Sasse, S.; Warnecke, A.; Lenarz, T.; Gross, G.; Hoffmann, A. Otology & neurotology: official publication of the American Otological Society, American Neurotology Society [and] European Academy of Otology and Neurotology 2008, 29, 475; (b) Toth, G.; Yang, H.; Anguelov, R. A.; Vettraino, J.; Wang, Y.; Acsadi, G. Journal of Neuroscience Research 2002, 69, 622.
(22) Gong, L.; Cai, Y.; Zhou, X.; Yang, H. Pathology and Oncology Research 2012, 18, 989.
(23) Monzavi-Karbassi, B.; Stanley, J. S.; Hennings, L.; Jousheghany, F.; Artaud, C.; Shaaf, S.; Kieber-Emmons, T.; International Journal of Cancer 2007, 120, 1179.
(24) Cooney, C. A; Jousheghany, F.; Yao-Borengasser, A.; Phanavanh, B.; Gomes, T.; Kieber-Emmons, A. M.; Siegel E. R., Suva, L. J.; Ferrone, S.; Kieber-Emmons, T.; Monzavi-Karbassi, B. Breast Cancer Research 2011, 13, R58.

1.-74. (canceled)

75. A non-saccharide glycosaminoglycan mimetic molecule comprising:
a polyproline backbone and one or more non-saccharide molecules;
wherein the non-saccharide molecules comprise primary, secondary or tertiary negatively charged groups, or a combination thereof; and
wherein the negatively charged groups are selected from the group consisting of sulfates, and phosphates.

76. The non-saccharide glycosaminoglycan mimetic molecule of claim 75, wherein the non-saccharide glycosaminoglycan mimetic molecule is one or more of the following:
(a) a non-saccharide glycosaminoglycan mimetic molecule wherein each proline in the polyproline backbone is independently a proline or a proline derivative;
(b) a non-saccharide glycosaminoglycan mimetic molecule, wherein the proline derivative comprises a functional group for conjugation to the non-saccharide molecule, wherein the proline derivative is optionally selected from the group consisting of azidoproline, aminoproline, mercaptoproline, prolinecarboxylic acid, hydroxyproline and enantiomers thereof;
(c) a non-saccharide glycosaminoglycan mimetic molecule, wherein the non-saccharide molecules have a structure selected from the group consisting of: 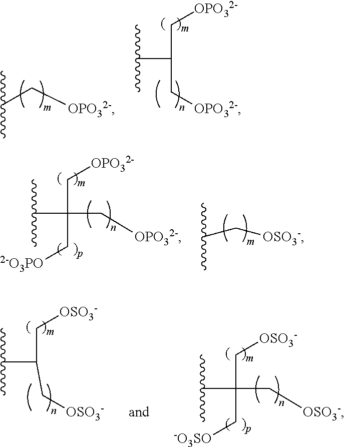
wherein m, n, and p are 0 or a positive integer greater than 1;
(d) a non-saccharide glycosaminoglycan mimetic molecule, wherein the non-saccharide molecules have the following structure: 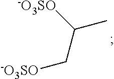
and
(e) a non-saccharide glycosaminoglycan mimetic molecule wherein the non-saccharide molecules are bound to one or more prolines and/or proline derivatives on the polyproline backbone.

77. The non-saccharide glycosaminoglycan mimetic molecule of claim 75, wherein the polyproline backbone has the following general formula (I): 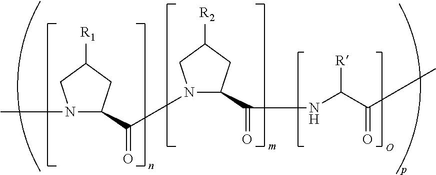
wherein R₁ and R₂ is H or a functional group for conjugation to a non-saccharide molecule; R′ is any amino acid side chain, n is a positive integer greater than 1; m is 0 or a positive integer, O is 0 or a positive integer, wherein at least one of R₁ or R₂ is a functional group for conjugation to a non-saccharide molecule, and p is a positive integer greater than 1.

78. The non-saccharide glycosaminoglycan mimetic molecule of claim 77, wherein the general formula (I) of the polyproline backbone is one or more of the following:
(a) a general formula (I) wherein n is 1, 2, 3, 4, 5, 6, 7, 8, 9, 10, 11 or 12;
(b) a general formula (I) wherein m is 0, 1, 2, 3, 4, 5, 6, 7, 8, 9, 10, 11 or 12;
(c) a general formula (I) wherein n is 1, m is 0, O is 0, R₁ is an azido group, and p is 12;
(d) a general formula (I) wherein n is 2, m is 1, O is 0, R₁ is H, R₂ is azido, and p is 12; and
(e) a general formula (I) wherein n is 4, m is 0, and 0 is 1, R₁ is azido, R′ is H, and p is 3.

79. The non-saccharide glycosaminoglycan mimetic molecule of claim 75, wherein the polyproline backbone has the following formula (II): 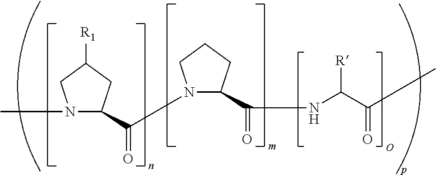
wherein R₁ is a functional group for conjugation to a non-saccharide molecule; R′ is any amino acid side chain; n is a positive integer greater than 1; m is 0 or a positive integer; O is 0 or a positive integer, and p is a positive integer greater than 1.

80. The non-saccharide glycosaminoglycan mimetic molecule of claim 75, wherein the non-saccharide glycosaminoglycan mimetic molecule is one or more of the following:
(a) a non-saccharide glycosaminoglycan mimetic molecule wherein the functional group for conjugation to a non-saccharide molecule is N3;
(b) a non-saccharide glycosaminoglycan mimetic molecule wherein the polyproline backbone comprises one or more glycine;
(c) a non-saccharide glycosaminoglycan mimetic molecule comprising more than one non-saccharide molecules; and
(d) a non-saccharide glycosaminoglycan mimetic molecule capable of binding cell adhesion molecules.

81. The non-saccharide glycosaminoglycan mimetic molecule of claim 75, wherein the non-saccharide molecules are attached at pre-determined positions along the polyproline backbone at one or more of the following positions:
at equal distances along the polyproline backbone;
along more than one different faces of the polyproline backbone;
along three faces and project from the polyproline backbone; and
along the same face of the polyproline backbone.

82. The non-saccharide glycosaminoglycan mimetic molecule of claim 75, further comprising one or more of the following:
polyethylene glycol (PEG) at one end of the polyproline backbone,
a biotin conjugated PEG at one end of the polyproline backbone,
a 1,2-dipalmitoyl-sn-glycero-3-phosphoethanolamine-N-(succinyl) conjugated PEG at one end of the polyproline backbone, and
a lipid.

83. The non-saccharide glycosaminoglycan mimetic molecule of claim 82, wherein the non-saccharide glycosaminoglycan mimetic molecule has the following general formula (III): 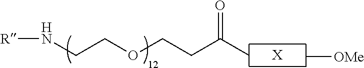
wherein R″ is any functional moiety, and wherein X is a non-saccharide glycosaminoglycan mimetic molecule comprising:
a polyproline backbone and one or more non-saccharide molecules;
wherein the non-saccharide molecules comprise primary, secondary or tertiary negatively charged groups, or a combination thereof; and
wherein the negatively charged groups are selected from the group consisting of sulfates, and phosphates.

84. The non-saccharide glycosaminoglycan mimetic molecule of claim 83, wherein the general formula (III) of the non-saccharide glycosaminoglycan mimetic molecule is one or more of the following:
(a) a general formula (III) wherein R″ is selected from the group consisting of H, 1,2-dipalmitoyl-sn-glycero-3-phosphoethanolamine-N-(succinyl)] and biotin; and
(b) a general formula (III) wherein X is (P_{E})₁₂, (PPP_{E})₁₂, or (P_{E})₄G(P_{E})₄G(P_{E})₄G wherein P is proline; G is glycine; and P_{E} is

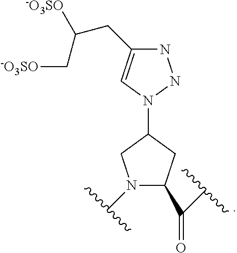

85. A method of synthesizing a non-saccharide glycosaminoglycan mimetic molecule according to claim 75, comprising attaching one or more non-saccharide molecules to a polyproline backbone.

86. The method of claim 85, wherein the method comprises one or more of the following:
(a) a method wherein the non-saccharide molecules comprise sulfated groups;
(b) a method wherein the non-saccharide molecules have the structure of 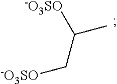
(c) a method wherein the non-saccharide molecules are alkyne-functionalized; and
(d) a method wherein the polyproline backbone is azido-functionalized.

87. The method of claim 85, wherein the one or more non-saccharide molecules are attached to the polyproline backbone via click reaction.

88. The method of claim 87, comprising one or more of the following steps:
conducting the click reaction in dimethyl sulfoxide (DMSO) at ambient temperature for 14 days in the presence of copper(I) idode, N,N-diisopropylethylamine (DIPEA) and tris[(1-benzyl-1H-1,2,3-triazol-4-yl)methyl]amine (TBTA) under argon atmosphere;
precipitating the reaction mixture resulting from the click reaction from a THF/methanol mixture and removing the solvent of the reaction mixture by vacuum and decanting the solid;
converting the reaction mixture into their sodium salt form; and
purifying the salt by size-exclusion chromatography.

89. The method of claim 88, wherein prior to the click reaction, the method comprises one or more of the following steps:
(a) conjugating PEG₁₂ to the polyproline backbone in the presence of N,N diisopropylethylamine (DIPEA) base and Dimethylformamide (DMF) at room temperature, and
(b) coupling 1,2-dipalmitoyl-sn-glycero-3-phosphoethanolamine-N-(succinyl) sodium salt or biotin to the PEG₁₂ via amide coupling in the presence of benzotriazol-1-yl-oxytripyrrolidinophosphonium hexafluorophosphate(PyBOB), hydroxybenzotriazole (HOBt), N,N-diisopropylethylamine (DIPEA) base and Dimethylformamide (DMF) at room temperature.

90. The method of claim 85, wherein the method comprises one or more of the following:
(a) a method wherein the non-saccharide molecules are alkyne-functionalized having the following structure: 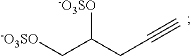
(b) a method wherein 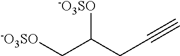
is synthesized by one or more of the following operations:
(i) reacting ethyl glyoxylate, 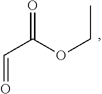
and propargyl bromide, 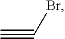
to form Ethyl 2-hydroxypent-4-ynoate, 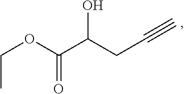
via a zinc Barbier reaction;
(ii) reducing the (Ethyl 2-hydroxypent-4-ynoate), 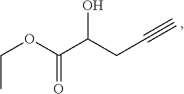
by lithium aluminum hydride to form Pent-4-yne-1,2-diol), 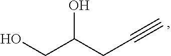
and
(iii) sulfating (Pent-4-yne-1,2-diol), 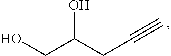
with an SO3.trimethyllamine complex (Sulfur trioxide trimethylamine complex) to form (Pent-4-yne-1,2-diyl bis(sulfate), 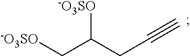
(c) a method wherein the Barbier reaction in operation (i) in (b) is conducted in the presence of zinc (Zn), calcium chloride (CaCl₂), ammonium chloride (NH₄Cl) and tetrahydrofuran-water (THF-H₂O) at room temperature;
(d) a method wherein operation (ii) in (b) is conducted in the presence of tetrahydrofuran (THF) at room temperature; and
(e) a method wherein operation (iii) in (b) is conducted in the presence of dimethylformamide (DMF) at room temperature.

91. A method selected from:
(a) a method for inhibiting cell adhesion molecules;
(b) a method of treating a patient in need of a target-specific therapy;
(c) a method of promoting neuritogenesis in a patient;
(d) a method of inhibiting extravasation of circulating tumor cell into potential metastatic sites in a patient;
(e) a method of treating a viral infection; and
(f) a method of treating a malaria infection;
comprising administering a non-saccharide glycosaminoglycan mimetic molecule comprising:
a polyproline backbone and one or more non-saccharide molecules;
wherein the non-saccharide molecules comprise primary, secondary or tertiary negatively charged groups, or a combination thereof; and
wherein the negatively charged groups are selected from the group consisting of sulfates, and phosphates.

92. The method of claim 91, wherein:
(i) the patient in the method of (b) is one suffering from a disease selected from Parkinson's disease, Alzheimer's disease and cancer;
(ii) the patient in the method of (c) is one suffering from a neurodegenerative disease selected from Alzheimer's disease, Parkinson's disease and Huntington's disease; and
(iii) the patient in the method of (d) is one suffering from a cancer selected from biliary tract cancer, brain cancer, breast cancer, cervical cancer, choriocarcinoma, colon cancer, endometrial cancer, esophageal cancer, gastric cancer, intraepithelial neoplasms, lymphomas, liver cancer, lung cancer (e.g. small cell and non-small cell), melanoma, neuroblastomas, oral cancer, ovarian cancer, pancreas cancer, prostate cancer, rectal cancer, sarcomas, skin cancer, testicular cancer, thyroid cancer, and renal cancer.

93. A method of controlling the binding affinity of a non-saccharide glycosaminoglycan mimetic molecule to one or more binding molecules, comprising:
attaching one or more non-saccharide molecules at pre-determined positions along a polyproline backbone;
wherein the non-saccharide molecules comprise primary, secondary or tertiary negatively charged groups, or a combination thereof; and
wherein the negatively charged groups are selected from the group consisting of sulfates, and phosphates.

94. The method of claim 93, wherein the method comprises one or more of the following:
(a) a method wherein each proline in the polyproline backbone is independently a proline or a proline derivative;
(b) a method wherein the proline derivative comprises a functional group for conjugation to a non-saccharide molecule, wherein the proline derivative is optionally selected from the group consisting of azidoproline, aminoproline, mercaptoproline, prolinecarboxylic acid, hydroxyproline and enantiomers thereof;
(c) a method wherein the non-saccharide molecules are defined as follows:
(i) the non-saccharide molecules have a structure selected from the group consisting of: 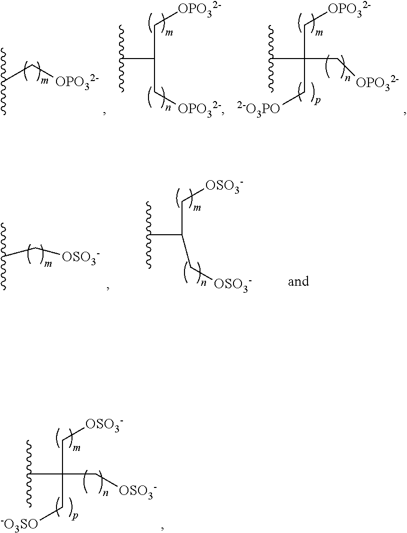
wherein m, n, and p are 0 or a positive integer greater than 1;
(ii) the non-saccharide molecules have the following structure: 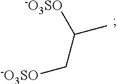
and
(iii) the non-saccharide molecules are bound to one or more prolines and/or proline derivatives on the polyproline backbone;
(d) a method wherein the polyproline backbone has the following general formula (I): 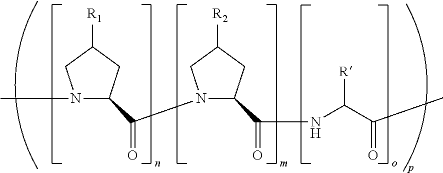
wherein R₁ and R₂ is H or a functional group for conjugation to a non-saccharide molecule; R′ is any amino acid side chain, n is a positive integer greater than 1; m is 0 or a positive integer, O is 0 or a positive integer, wherein at least one of R₁ or R₂ is a functional group for conjugation to a non-saccharide molecule, and p is a positive integer greater than 1;
(e) a method comprising attaching the non-saccharide molecules at pre-determined positions along the polyproline backbone at one or more of the following positions:
at equal distances from each other along the polyproline backbone,
along more than one different faces of the polyproline backbone,
along three faces and project from the polyproline backbone, and
along the same face of the polyproline backbone; and
(f) a method wherein the non-saccharide glycosaminoglycan mimetic molecule comprises more than one non-saccharide molecules.
